SEQUENCE LISTING (30 sequences)

SEQ ID NO: 1 - protein, 6 aa, Homo sapiens
  REGION 1..6: note = Human laminin-1-derived peptide
SIKVAV

SEQ ID NO: 2 - protein, 581 aa, Human adenovirus type 5
  REGION 1..581: note = Human adenovirus type 5 fiber ACCESSION NO: AP_000226
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 3 - protein, 325 aa, Human adenovirus type 11
  REGION 1..325: note = Human adenovirus type 11 fiber ACCESSION NO: AAA42490
MTKRVRLSDSFNPVYPYEDESTSQHPFINPGFISPNGFTQSPNGVLTLKCLTPLTTTGGSLQLKVGGGLTVDDTNGFLKENISATTPLVKTGHSIGLPLGAGLGTNENKLCIKLGQGLTFNSNNICIDDNINTLWTGVNPTEANCQIMNSSESNDCKLILTLVKTGALVTAFVYVIGVSNNFNMLTTHRNINFTAELFFDSTGNLLTRLSSLKTPLNHKSGQNMATGAITNAKGFMPSTTAYPFNDNSREKENYIYGTCYYTASDRTAFPIDISVMLNRRAINDETSYCIRITWSWNTGDAPEVQTSATTLVTSPFTFYYIREDD

SEQ ID NO: 4 - protein, 353 aa, Human adenovirus type 16
  REGION 1..353: note = Human adenovirus type 16 fiber ACCESSION NO: AET87329
MAKRARLSSSFNPVYPYEDESSSQHPFINPGFISSNGFAQSPDGVLTLKCVNPLTTASGPLQLKVGSSLTVDTIDGSLEENITAAAPLTKTNHSIGLSIGSGLQTKDDELCLSLGDGLVTKDDKLCLSLGDGLITKDDTLCAKLGHGLVFDSSNAITIENNTLWTGAKPSANCVIQEGEDSPDCKLTLVLVKNGGLINGYITLMGASQYTNTLFKNKQVTINVNLAFDNTGQIITYLSSLKSNLNFKDNQNMATGTITSAKGFMPSTTAYPFTIYATQSLNEDYIYGECYYKSTNGTLFPLKVTVTLNRRMSASGMAYAMNFSWSLNAEQAPETTEVTLITSPFFFSYIREDD

SEQ ID NO: 5 - protein, 323 aa, Human adenovirus type 21
  REGION 1..323: note = Human adenovirus type 21 fiber ACCESSION NO: AAW33370
MTKRVRLSDSFNPVYPYEDESTSQHPFINPGFISPNGFTQSPDGVLTLNCLTPLTTTGGPLQLKVGGGLIVDDTDGTLQENIRATAPITKNNHSVELSIGNGLETQNNKLCAKLGNGLKFNNGDICIKDSINTLWTGIKPPPNCQIVENTDTNDGKLTLVLVKNGGLVNGYVSLVGVSDTVNQMFTQKSATIQLRLYFDSSGNLLTDESNLKIPLKNKSSTATSEAATSSKAFMPSTTAYPFNTTTRDSENYIHGICYYMTSYDRSLVPLNISIMLNSRTISSNVAYAIQFEWNLNAKESPESNIATLTTSPFFFSYIREDDN

SEQ ID NO: 6 - protein, 323 aa, Human adenovirus type 34
  REGION 1..323: note = Human adenovirus type 34 fiber ACCESSION NO: AAW33501
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTDESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 7 - protein, 323 aa, Human adenovirus type 35
  REGION 1..323: note = Human adenovirus type 35 fiber ACCESSION NO: AP_000601
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 8 - protein, 323 aa, Human adenovirus type 50
  REGION 1..323: note = Human adenovirus type 50 fiber ACCESSION NO: AAW33547
MTKRVRLSDSFNPVYPYEDESTSQHPFINPGFISPNGFTQSPDGVLTLNCLTPLTTTGGPLQLKVGGGLIVDDTDGTLQENIRVTAPITKNNHSVELSIGNGLETQNNKLCAKLGNGLKFNNGDICIKDSINTLWTGIKPPPNCQIVENTDTNDGKLTLVLVKNGGLVNGYVSLVGVSDTVNQMFTQKSATIQLRLYFDSSGNLLTDESNLKIPLKNKSSTATSEAATSSKAFMPSTTAYPFNTTTRDSENYIHGICYYMTSYDRSLVPLNISIMLNSRTISSNVAYAIQFEWNLNAKESPESNIATLTTSPFFFSYIREDDN

SEQ ID NO: 9 - protein, 319 aa, Human adenovirus type 3
  REGION 1..319: note = Human adenovirus type 3 fiber ACCESSION NO: ABB17809
MAKRARLSTSFNPVYPYEDESSSQHPFINPGFISPDGFTQSPNGVLSLKCVNPLTTASGSLQLKVGSGLTVDTTDGSLEENIKVNTPLTKSNHSINLPIGNGLQIEQNKLCSKLGNGLTFDSSNSIALKNNTLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDD

SEQ ID NO: 10 - protein, 325 aa, Human adenovirus type 7
  REGION 1..325: note = Human adenovirus type 7 fiber ACCESSION NO: QEQ50315
MTKRVRLSDSFNPVYPYEDESTSQHPFINPGFISPNGFTQSPDGVLTLKCLTPLTTTGGSLQLKVGGGLTIDDTDGFLKENISATTPLVKTGHSIGLSLGPGLETNENKLCAKLGEGLTFNSNNICINDNINTLWTGVNPTRANCQIMASSESNDCKLILTLVKTGALVTAFVYVIGVSNDFNMLTTHKNINFTAELFFDSTGNLLTSLSSLKTPLNHKSGQNMATGALTNAKGFMPSTTAYPFNVNSREKENYIYGTCYYTASDHTAFPIDTSVMLNQRALNNETSYCIRVTWSWNTGVAPEVQTSATTLVTSPFTFYYIREDD

SEQ ID NO: 11 - protein, 323 aa, Human adenovirus type 14
  REGION 1..323: note = Human adenovirus type 14 fiber ACCESSION NO: ACO81814
MTKRVRLSDSFNPVYPYEDESTSQHPFINPGFISPNGFTQSPDGVLTLKCLTPLTTTGGSLQLKVGGGLTVDDTDGTLQENIGATTPLVKTGHSIGLSLGAGLGTDENKLCTKLGEGLTFNSNNICIDDNINTLWTGVNPTEANCQMMDSSESNDCKLILTLVKTGALVTAFVYVIGVSNNFNMLTTYRNINFTAELFFDSAGNLLTSLSSLKTPLNHKSGQNMATGAITNAKSFMPSTTAYPFNNNSRENYIYGTCHYTASDHTAFPIDISVMLNQRAIRADTSYCIRITWSWNTGDAPEGQTSATTLVTSPFTFYYIREDD

SEQ ID NO: 12 - protein, 325 aa, Human adenovirus type 55
  REGION 1..325: note = Human adenovirus type 55 fiber ACCESSION NO: AQY04135
MTKRVRLSDSFNPVYPYEDESTSQHPFINPGFISPNGFTQSPDGVLTLKCLTPLTTTGGSLQLKVGGGLTVDDTDGTLQENIGTTTPLVKTGHSIGLSLGAGLGTDENKLCTKLGKGLTFNSNNICIDDNINTLWTGINPTEANCQMMDSSESNDCKLILTLVKTGALVTAFVYVIGVSNNFNMLTTYRNINFTAELFFDSAGNLLTSLSSLKTPLNHKSGQNMATGAITNAKSFMPSTTAYPFNNNSREKENYIYGTCHYTASDHTAFPIDISVMLNQRAIRADTSYCIRITWSWNTGDAPEGQTSATTLVTSPFTFYYIREDD

SEQ ID NO: 13 - protein, 437 aa, synthetic construct
  REGION 1..437: note = Ad5/35 fiber, chimeric artificial
MKRARPSEDTFNPVYPYDTETGPPTVPFLTPPFVSPNGFQESPPGVLSLRLADPVTTKNGEITLKLGEGVDLDDSGKLIANTVNKAIAPLSFSNNTISLNMDTPLYTKDGKLSLQVSPPLSILKSTILNTLALAFGSGLGLSGSALAVQLASPLTFDDKGNIKITLNRGLHVTTGDAIESNISWAKGIKFEDGAIATNIGKGLEFGTSSTETGVNNAYPIQVKLGSGLSFDSTGAIMAGNKDYDKLTLWTGINPPPNCQIVENTNTNDGKLTLVLVKNGGLVNGYVSLVGVSDTVNQMFTQKTANIQLRLYFDSSGNLLTDESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 14 - protein, 439 aa, synthetic construct
  REGION 1..439: note = Ad5/14 fiber, chimeric artificial, Fiber mutant; Artificial
MKRARPSEDTFNPVYPYDTETGPPTVPFLTPPFVSPNGFQESPPGVLSLRLADPVTTKNGEITLKLGEGVDLDDSGKLIANTVNKAIAPLSFSNNTISLNMDTPLYTKDGKLSLQVSPPLSILKSTILNTLALAFGSGLGLSGSALAVQLASPLTFDDKGNIKITLNRGLHVTTGDAIESNISWAKGIKFEDGAIATNIGKGLEFGTSSTETGVNNAYPIQVKLGSGLSFDSTGAIMAGNKDYDKLTLWTGVNPTEANCQMMDSSESNDCKLILTLVKTGALVTAFVYVIGVSNNFNMLTTYRNINFTAELFFDSAGNLLTSLSSLKTPLNHKSGQNMATGAITNAKSFMPSTTAYPFSNNSREKENYIYGTCHYTASDHTAFPIDISVMLNQRAIRADTSYCIRITWSWNTGDAPEGQTSATTLVTSPFTFYYIREDD

SEQ ID NO: 15 - skipped

SEQ ID NO: 16 - protein, 11 aa, Human adenovirus type 5
  REGION 1..11: note = Human adenovirus type 5 penton base RGD loop sequence, partial (FIG. 7A)
DHAIRGDTFAT

SEQ ID NO: 17 - protein, 571 aa, Human adenovirus type 5
  REGION 1..571: note = Human adenovirus type 5 penton ACCESSION NO: AAA42519
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 18 - protein, 11 aa, synthetic construct
  REGION 1..11: note = Human laminin-1-derived peptide, artificial (FIG. 8A)
AASIKVAVSAA

SEQ ID NO: 19 - protein, 507 aa, synthetic construct
  REGION 1..507: note = Penton mutant; Artificial (FIG. 8B)
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDAASIKVAVSAAKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 20 - protein, 507 aa, synthetic construct
  REGION 1..507: note = Penton mutant; Artificial
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVTAYQASLKETAASIKVAVSAAKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 21 - protein, 12 aa, synthetic construct
  REGION 1..12: note = Alpha4-integrin-interacting peptide, artificial (FIG. 9A)
AAEILDVPSTAA

SEQ ID NO: 22 - protein, 508 aa, synthetic construct
  REGION 1..508: note = Penton mutant; Artificial (FIG. 9B)
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDAAEILDVPSTAAKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 23 - protein, 12 aa, synthetic construct
  REGION 1..12: note = Alpha9-integrin-interacting peptide, artificial (FIG. 10A)
AAAEIDGIELAA

SEQ ID NO: 24 - protein, 508 aa, synthetic construct
  REGION 1..508: note = Penton mutant; Artificial (FIG. 10B)
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDAAAEIDGIELAAKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 25 - protein, 12 aa, synthetic construct
  REGION 1..12: note = AlphaD-integrin-interacting peptide, artificial (FIG. 11A)
AATQIDSPLNAA

SEQ ID NO: 26 - protein, 508 aa, synthetic construct
  REGION 1..508: note = Penton mutant; Artificial (FIG. 11B)
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDAATQIDSPLNAAKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 27 - protein, 202 aa, Homo sapiens
  REGION 1..202: note = Shaft domain of the fiber protein, derived from human adenovirus serotype HAdv-E4.
LSLRLADPVTTKNGEITLKLGEGVDLDDSGKLIANTVNKAIAPLSFSNNTISLNMDTPLYTKDGKLSLQVSPPLSILKSTILNTLALAFGSGLGLSGSALAVQLASPLTFDDKGNIKITLNRGLHVTTGDAIESNISWAKGIKFEDGAIATNIGKGLEFGTSSTETGVNNAYPIQVKLGSGLSFDSTGAIMAGNKDYDKLTL

SEQ ID NO: 28 - protein, 8 aa, Homo sapiens
  REGION 1..8: note = Alpha-4-integrin-interacting peptide, human fibronectin-derived
EILDVPST

SEQ ID NO: 29 - protein, 8 aa, synthetic construct
  REGION 1..8: note = Alpha-9-integrin-interacting peptide, artificial
AEIDGIEL

SEQ ID NO: 30 - protein, 8 aa, synthetic construct
  REGION 1..8: note = Alpha-D-integrin-interacting peptide, artificial
TQIDSPLN

(19) United States
(12) Patent Application Publication
(10) Pub. No.: US 2025/0186611 A1
(43) Pub. Date: Jun. 12, 2025

(54) MULTIPLEXED-TARGETED ADENOVIRUS VECTORS AND THEIR USE

(71) Applicants: Adcure Bio, LLC, Snellville, GA (US); Emory University, Atlanta, GA (US)

(72) Inventors: Dmitry M. SHAYAKHMETOV, Snellville, GA (US); Nelson C. DI PAOLO, Everett, WA (US)

(21) Appl. No.: 18/665,155

(22) Filed: May 15, 2024

Related U.S. Application Data
(63) Continuation-in-part of application No. 18/633,113, filed on Apr. 11, 2024, now abandoned.
(60) Provisional application No. 63/497,106, filed on Apr. 19, 2023.
(60) Provisional application No. 63/640,520, filed on Apr. 30, 2024.

(51) Int. Cl. A61K 48/00; C07K 14/705; C12N 15/86

(52) U.S. Cl. CPC A61K 48/0041; C07K 14/70596; C12N 15/86; C12N 2710/10022; C12N 2710/10043

(57) ABSTRACT

The present application provides compositions and methods for increasing safety, selectivity, and efficacy of transduction of human cells, including human long-term repopulating and hematopoietic stem cells (LT-HSC) and human cancer cells, by adenovirus vectors through multiplexed targeting of virus attachment and internalization receptors. Multiplexing targeting Ad vector receptor specificities through the combination of i) restricting fiber-specific attachment receptors, ii) deleting the RGD amino acid motif from the penton base protein, and iii) inserting into Ad penton base protein of peptides that lack the RGD amino acid motif and enable vector interaction with integrin classes expressed on target cells, allows for the improvement of safety, selectivity, and efficacy of vector-mediated transduction of human cells in vitro and after intravenous vector administration in vivo.

This application is a continuation-in-part application of U.S. application Ser. No. 18/633,113, filed Apr. 11, 2024, which claims priority from U.S. Provisional Application No. 63/497,106, filed Apr. 19, 2023. The entirety of the aforementioned applications are incorporated herein by reference.

This application also claims priority to Provisional Application No. 63/640,520, filed Apr. 30, 2024. The entirety of the aforementioned application is incorporated herein by reference.

This invention was made with government support under Grant No. AI107960 awarded by the National Institutes of Health. The government has certain rights in the invention.

### FIELD

The present application relates to the field of medicine, in particular the fields of gene therapy and cancer therapy, where adenovirus vectors with modified capsids are administered to patients with genetic or acquired diseases for therapeutic purposes to attain desired therapeutic benefits.

### BACKGROUND

Adenovirus is a ubiquitous pathogen causing a wide range of human diseases, which include respiratory tract infections, conjunctivitis, hemorrhagic cystitis, and gastro-intestinal diseases. In immunocompetent patients, Ad infections are self-limited, and after resolution of the acute infection, the virus remains latent in lymphoid and renal tissues. In contrast, in immunocompromised patients Ad infections may cause life threatening or even fatal fulminant hepatitis and disseminated infection of other tissues.

There are currently over 60 characterized human Ad serotypes which are divided into seven “species” (formally subgroups) from A to G. Although various Ad serotypes may initiate infections via different transmission routes, and utilize distinct virus attachment receptors, the host factors and cell types controlling tissue specificity of Ad infection in vivo remain insufficiently understood.

The Ad infectious cycle occurs in two steps. The early phase precedes the initiation of replication and makes it possible to produce the early proteins regulating the replication and transcription of the viral DNA. The replication of the genome is followed by the late phase during which the structural proteins that constitute the viral particles are synthesized. The assembly of the new virions takes place in the host cell nucleus. In a first stage, the viral proteins assemble to form empty capsids of icosahedral structure into which the genome is encapsidated. The assembled virus includes hexon, a penton base, fiber, and other minor proteins. The Ads liberated from cells are then capable of infecting other permissive cells. The fiber and the penton base proteins present at the surface of the capsids play a role in the cellular attachment of the virions and their internalization.

In vitro studies have demonstrated that Ad infection starts with the virus binding to a high affinity primary attachment receptor on the cell surface. The trimeric Ad fiber protein mediates this interaction when its distal knob domain binds to a specific cellular receptor. For binding to cells, species A, C, D, E, and F human Ads may utilize the coxsackievirus and Ad receptor (CAR); however, human species B Ads utilize CD46 or DSG2 as high affinity cellular attachment receptors. It is established in the art that substitution of the fiber knob domain in adenovirus that utilizes one of the high affinity attachment receptors for the fiber knob domain derived from another serotype that utilizes a different attachment receptor enables re-targeting of such fiber-mutated virus to a different, non-cognate attachment receptor.

It is also established in the art that adenovirus vector-mediated gene transfer in vivo is not governed exclusively by the specificity of the fiber knob domain. It is further established that the selectivity of gene transfer to a desired cell type in vivo after intravenous administration of adenovirus vector cannot be predicted a priori without extensive experimentation. All three high affinity adenovirus attachment receptors, CAR, CD46, and DSG2, are broadly expressed in vivo on a variety of human cells and the utility of one or the other receptor as a targeting moiety that may be most suitable to mediate cell type specific adenovirus vector-mediated gene delivery in vivo without undesired and toxic side effects cannot be reliably predicted or determined without experimentation.

Fiber-mediated binding of Ad to cells is followed by the RGD amino acid motif-mediated binding of the viral penton base protein to cellular integrins of RGD-interacting classes, primarily but not limited to αvβ3 and αvβ5 classes. This interaction induces integrin activation and cytoskeleton rearrangement that facilitates internalization of the virus particle into the cell.

Cells express a variety of alpha and beta class integrins on their surfaces, which mediate cell attachment to different extracellular matrix proteins. The type of integrins expressed on a particular cell type defines its ability to traffic through the body to and reside in an appropriate niche within a specific tissue according to their relevant function(s). Particular selectivities exist in integrin classes associated with particular cell types in order to support diverse cellular functions, including ability for attachment to various extracellular matrices in different tissue microenvironments and different physiological or pathological contexts. For instance, human airway epithelial cells express eight different integrin heterodimers, including a2b1, a3b1, a6b4, a9b1, a5b1, avb5, avb6, avb8. These heterodimers recognize collagen I, tenascin C, laminins 5, 10, and 11, osteopontin, fibronectin, vitronectin, and others extracellular matrix proteins. Human NK cells circulating in the blood express αLβ2, αMβ2, α4β1, α5β1, and α6β1 integrins. However, homing of NK cells to tissues is associated with upregulated expression of albl and aEb2 integrin classes. Human melanoma tumor cells express a4b1, a2b1, and avb3 integrin classes, which drive differential migration and homing of tumor cells to distant organs, e.g., brain, liver, lung, bone tissue, and lymph nodes. Furthermore, human high-grade gliomas are known to upregulate expression of a2b1, a3b1, a5b1, a4b1, and a6b1 integrin classes, while expression of avb3 and avb5 integrins on tumor-associated endothelial cells is known to correlate with tumor progression.

Although a variety of integrin-targeting peptides, antibodies, and small molecule drugs have been developed to improve selectivity of therapy delivery to specific cell types in vivo, numerous clinical trials have failed to demonstrate sufficient therapeutic efficacy of this approach. Factors attributing to such integrin-targeting therapeutic failures include e.g., variable integrin expression on target cells and the redundancy in function of different classes of integrins. It is also clear that the expression of a variety of integrin classes with promiscuous and redundant ligand specificities on the same cell type further complicates approaches for utilizing specific integrin classes for cell type-specific targeting and therapy delivery. Although cell type-specific therapy delivery remains a highly desirable property for viral gene therapy vectors, non-viral gene therapy delivery platforms, as well as viral and non-viral cancer therapeutics, the poor clinical trial results suggest that integrins are unlikely to be useful molecules enabling cell type-specific targeting of therapy and that a success in improving selectivity of drug delivery to specific cell types via integrins is not guaranteed and cannot be predicted without extensive experimentation. To date, cell type-specific targeting for therapeutic delivery, especially after intravenous administration, remains a challenging area of translational research and drug development.

Whereas natural adenovirus infections in humans rarely cause severe pathology, intravenous injection of adenovirus-based vectors, especially at high doses, triggers rapid activation of the innate immune system, leading to cytokine storm syndrome, disseminated intravascular coagulation, thrombocytopenia, and hepatotoxicity, which individually or in combination may cause morbidity and mortality. It is known in the art that Ad sequestration in liver resident macrophages, Kupffer cells, and macrophages residing in other organs of the body, such as spleen, is the principal step leading to systemic virus-associated toxicity due to activation of innate immune mechanisms of host defense, which recognize therapeutic Ad vector as a pathogen. It is established in the art that the RGD amino acids within the RGD loop of Ad penton base bind to macrophage integrins of b3 class, and this binding activates expression and release of IL-1a and other inflammatory cytokines and chemokines in liver and spleen through IL-1-IL-1RI signaling feed forward amplification loop. Specifically, adenovirus vectors do not trigger pro-inflammatory cytokine production after intravenous administration if such vectors lack the RGD amino acids in the penton or if mice lack b3 integrins.

Although adenovirus vectors with deleted or mutated RGD amino acid motif in the penton base trigger reduced inflammatory cytokine activation after intravenous administration, such vectors are inefficient at internalization and endosome escape steps of virus entry into cells and are considered inferior platforms for therapeutic gene transfer compared to vectors that possess intact RGD amino acid motif in the penton base protein.

In view of the foregoing, there is a need in the art for adenovirus vectors with improved receptor selectivity that would allow for a targeted cell-type-specific gene delivery in vitro and in vivo, while preserving the useful property of reduction in vector-associated toxicities, to enable clinical application of this vector platform for therapy of human diseases.

### BRIEF DESCRIPTION OF THE DRAWINGS

FIG. 1 is a structural depiction of an adenovirus (Ad) particle, including structural features of an Ad capsid reconstructed using cryo-electron microscopy. Arrows depict a fiber protein protruding from the Ad capsid, including localization of the penton base protein, which forms a homo-pentameric complex structurally linked to the fiber. The principal functions of the fiber and penton complex related to Ad attachment and entry into cells.

FIG. 2 shows receptor specificity for selected human adenovirus serotypes from specific human adenovirus species (designated from A to F) in terms of their cell attachment receptor usage conferred by the fiber protein, and utilization of the RGD interacting integrins by the penton protein to promote adenovirus internalization into cells. A select number of adenovirus serotypes are shown for each species family. The human species D family (denoted by the asterisk) is comprised of many individual adenovirus serotypes with fibers recognizing various cell attachment receptors. A select number of species D adenovirus serotypes is shown.

FIG. 3 is a schematic diagram of heterodimer complex formation between various □ and □ integrin species of different integrin classes, including RGD receptors, Laminin receptors, and Collagen receptors. Denoted by the star is integrin □9□1 that recognizes a variety of ligands, including VCAM-1, osteopontin, and tenascin-C.

FIG. 4A shows a UMAP plot showing annotation of human hematopoietic long-term repopulating and stem cells (LT-HSCs), expressing PROCR gene (shown as dark black dots, circled), among various primitive human progenitor CD34-positive cell populations (light gray). The annotation is based on analysis of single cell transcriptional profile data in individual cells present in human LT-HSC and early progenitor populations may be found in the ArrayExpress database under the dataset file number E-MTAB-9067 (Ranzoni et al., 2021). The dataset file number E-MTAB-9067 was uploaded into program R, and the UMAP map image was constructed using a Seurat statistical package in R.

FIG. 4B shows a distribution of cells (dark black dots) expressing the adenovirus attachment receptor CD46 in LT-HSC cells (circled) and other committed progenitor populations based on the analysis of single cell transcriptional profile data for individual cells. A UMAP image was generated as described in the legend to FIG. 4A. LT-HSC cells and all other progenitor cell populations were found to express CD46.

FIG. 4C shows a distribution of cells (dark black dots) expressing adenovirus attachment receptor DSG2 in LT-HSC cells (circled) and committed progenitor populations (light gray dots) based on the analysis of single cell transcriptional profile data for individual cells. A UMAP image was generated as described in the legend to FIG. 4A. LT-HSC cells were found to express the high levels of DSG2 compared to other progenitor cell populations.

FIG. 4D shows a distribution of cells (dark black dots) expressing adenovirus attachment receptor CXADR (CAR) in LT-HSC cells (circled) and committed progenitor populations (light gray) based on the analysis of single cell transcriptional profile data for individual cells. A UMAP image was generated as described in the legend to FIG. 4A. LT-HSC cells were found to express no or low levels of CXADR (CAR), while other progenitor cell populations were found to express high levels of CXADR.

FIG. 5A shows a distribution of cells (dark black dots) expressing integrin ITGB 1 gene in LT-HSC cells (circled) and committed progenitor populations (light gray) based on the analysis of single cell transcriptional profile data for individual cells. A UMAP image was generated as described in the legend to FIG. 4A. LT-HSC cells and all other progenitor cell populations were found to express the integrin □1 class member, encoded by ITGB1.

FIG. 5B shows distribution of cells expressing integrin ITGA5 gene (dark black dots) in LT-HSC cells (circled) and committed progenitor populations (light gray) based on the analysis of single cell transcriptional profile data for individual cells. A UMAP image was generated as described in the legend to FIG. 4A. LT-HSC cells and other progenitor cell populations were found to express high levels of the integrin □5 class member, encoded by ITGA5, which is known to interact with RGD motif-containing ligands.

FIG. 5C shows a distribution of cells expressing integrin ITGAV gene (dark black dots) in LT-HSC cells (circled) and committed progenitor populations (light gray) based on the analysis of single cell transcriptional profile data for individual cells. A UMAP image was generated as described in the legend to FIG. 4A. LT-HSC cells represent the major cell population, selectively expressing high levels of the integrin □V class member, encoded by ITGAV, which interacts with RGD motif-containing ligands.

FIG. 5D shows a distribution of cells expressing integrin ITGA6 gene (dark black dots) in LT-HSC cells (circled) and committed progenitor populations (light gray) based on the analysis of single cell transcriptional profile data for individual cells. A UMAP image was generated as described in the legend to FIG. 4A. Similar to the expression patter of ITGAV gene, that encodes RGD-interacting integrin classes, LT-HSC cells represent the major cell population expressing high levels of the integrin □6 class member, encoded by ITGA6.

FIG. 6A shows a schematic diagram of the domain structure of the adenovirus fiber protein. The fiber tail domain mediates anchoring of the fiber into the virus capsid through binding to the penton base-pentamer complex, localized within all vertices of the icosahedral virion particle. The fiber knob domain mediates binding to a virus attachment receptor at the cell surface. A rod-like fiber shaft domain mediates spatial separation between fiber knob and the tail domain and the protrusion of the fiber knob domain away from the virion surface.

FIG. 6B shows an exemplary domain structure of the adenovirus fiber interacting with CD46 or DSG2 attachment receptors, but not CAR, suitable for multiplexed vector targeting to enable it attachment to cells expressing these cell surface receptors.

FIG. 7A shows the amino acid sequence (SEQ ID NO: 16) of the RGD motif-containing region in the RGD loop of the human adenovirus HAdv-C5 penton base protein. The RGD amino acids (SEQ ID NO: 15) are underlined and highlighted in bold. Amino acid position numbers corresponding to the penton base protein sequence indicated as reported for the human adenovirus HAdv-C5 penton base protein amino acid sequence in the NCBI database (Accession number AAA42519).

FIG. 7B shows the complete amino acid sequence (SEQ ID NO: 17) of the human adenovirus HAdv-C5 penton base protein in the NCBI database (Accession number AAA42519). The RGD loop is underlined and the RGD amino acids (SEQ ID NO: 15) are highlighted in bold.

FIG. 8A shows an amino acid sequence of the artificial peptide AASIKVAVSAA (SEQ ID NO: 18) that comprises a human laminin-1-derived amino acid motif SIKVAV (SEQ ID NO: 1; underlined and highlighted in bold) that is known to interact with a6 class integrins and other laminin-interacting integrins but not with RGD interacting integrins.

FIG. 8B shows the artificial amino acid sequence (SEQ ID NO: 19) of the human adenovirus HAdv-C5 penton base protein comprising an insertion of the SEQ ID NO: 18 amino acids into the RGD loop region of the human adenovirus HAdv-C5 penton base protein. The functional SIKVAV amino acid motif (SEQ ID NO: 1) is underlined and the entire artificial SIKVAV amino acid motif-containing peptide is highlighted in bold.

FIG. 9A shows an amino acid sequence of the artificial peptide AAEILDVPSTAA (SEQ ID NO:21) that comprises amino acid motif EILDVPST (SEQ ID NO: 28; underlined and highlighted in bold) that is known to interact with a4-class integrins but not with RGD-interacting integrins.

FIG. 9B shows the artificial amino acid sequence (SEQ ID NO:22) of the human adenovirus HAdv-C5 penton base protein comprising an insertion of the SEQ ID NO:21 amino acids into the RGD loop region of the human adenovirus HAdv-C5 penton base protein. The functional EILDVPST amino acid motif (SEQ ID NO:28) is underlined and the entire artificial peptide inserted into the RGD loop of the penton base protein is highlighted in bold.

FIG. 10A shows an amino acid sequence of the artificial peptide AAAEIDGIELAA (SEQ ID NO:23) that comprises an amino acid motif AEIDGIEL (SEQ ID NO: 29; underlined and highlighted in bold) that is known to interact with a9-class integrins but not with RGD-interacting integrins.

FIG. 10B shows the artificial amino acid sequence (SEQ ID NO:24) of the human adenovirus HAdv-C5 penton base protein comprising an insertion of the SEQ ID NO:23 amino acids into the RGD loop region of the human adenovirus HAdv-C5 penton base protein. The functional AEIDGIEL amino acid motif (SEQ ID NO:29) is underlined and the entire artificial peptide inserted into the RGD loop of the penton base protein is highlighted in bold.

FIG. 11A shows an amino acid sequence of the artificial peptide AATQIDSPLNAA (SEQ ID NO:25) that comprises an amino acid motif TQIDSPLN (SEQ ID NO: 30; underlined and highlighted in bold) that is known to interact with aD class but not with RGD interacting integrins.

FIG. 11B shows the artificial amino acid sequence (SEQ ID NO:26) of the human adenovirus HAdv-C5 penton base protein comprising an insertion of the SEQ ID NO:25 amino acids into the RGD loop region of the human adenovirus HAdv-C5 penton base protein. The functional TQIDSPLN amino acid motif (SEQ ID NO:30) is underlined and the entire artificial peptide inserted into the RGD loop of the penton base protein is highlighted in bold.

FIG. 12 shows the curve plot of human lung adenocarcinoma A549 cells transduction by AVID-317-CMV-GFP virus, analyzed by flow cytometry 24 hours after cell infection. Each dot represents the experimentally determined percent of GFP-positive cells (% GFP+) determined upon infection of A549 cells with the virus at indicated MOIs. The curve fitting and EC50 calculations were performed using GraphPad Prism 9.4.0 software.

FIG. 13 shows the curve plot of human lung adenocarcinoma A549 cells transduction by AVID-388-CMV-GFP virus, analyzed by flow cytometry 24 hours after cell infection. Each dot represents the experimentally determined percent of GFP-positive cells (% GFP+) determined upon infection of A549 cells with the virus at indicated MOIs. The curve fitting and EC50 calculations were performed using GraphPad Prism 9.4.0 software.

FIGS. 14A-14D show the amounts of pro-inflammatory cytokines in plasma of mice after administration of indicated viruses. The amounts of pro-inflammatory cytokines IL-6 (FIG. 14A) TNF-α (FIG. 14B), IL-1b (FIG. 14C), and IFN-g (FIG. 14D) in the plasma of mice 6 hours after administration of Buffer (control group), unmodified wild type HAdv-B11 virus, AVID-317, and AVID-388 vectors with modified capsids. Twelve-week-old C57BL/6 mice were administered with indicated viruses or buffer intravenously into a tail vein in a total volume of 200 μl and 6 hours later, blood was collected through retro-orbital bleeding procedure into heparinized Eppendorf tubes. Plasma was immediately prepared, aliquoted, and stored at −80 C until use. The amounts of pro-inflammatory cytokine in plasma were determined using Bio-Rad Bio-Plex Pro™ Mouse Cytokine Panel 23-Plex #M60009RDPD in accordance with manufacturer's instructions using 25 μl of plasma sample for each mouse. Mice were administered in groups. N=5 for the Buffer group; N=5 for HAdv-B11 group; N=5 for AVID-317 group; and N=5 for AVID-388 group. All data are presented as mean±SD. *p<0.05, **p<0.01, ***p<0.001, and ****p<0.0001. Each dot represents results from one mouse.

FIG. 15 shows proportions of CD33-positive myeloid cells among human peripheral blood mononuclear cells (PBMCs) 24 hours after infection with indicated vectors. Human PBMCs from healthy donors were infected with Ad5/35, AVID-317, and AVID-388 at MOI 5000 virus particles per cell in vitro. Twenty-four hours post infection, cells were analyzed with flow cytometry to determine the percentages of CD33+ myeloid cells in total CD45+ cells. Frequencies of myeloid cell population were quantified, and t test was used to compare the proportion of myeloid cells in mock-infected samples (MOCK) and virus infected samples for each virus. n=4-5. All data are presented as mean±SD. *p<0.05, **p<0.01, ***p<0.001; ns, not significant.

FIG. 16 shows the dot plots of primary human CD34+ cells, isolated from mobilized peripheral blood, infected with AVID-388-CMV-GFP virus at an MOI of 5000 v.g. per cell or uninfected cells, stained with APC-labeled anti-CD34 antibodies, and analyzed 48 h after the virus infection by flow cytometry. The GFP-expressing CD34-positive cells are shifted upward long the Y-axis (GFP) and designated by a rectangle. Percent of GFP-positive cells after virus infection or in uninfected cells is shown on the corresponding dot plots. For this analysis, CD34+ cells were gated out of live and single cells, prior to analyzing percent GFP positive cells.

FIG. 17 shows the analysis of primary human CD34+CD38-hematopoietic stem and progenitor cell (HSPCs) infection with AVID-388-CMV-GFP virus. The dot plots show the data obtained upon infection of primary human CD34+ cells with AVID-388-CMV-GFP virus at an MOI of 5000 v.g. per cell or uninfected cells, stained with APC-labeled anti-CD34 and PerCP-labeled anti-CD38 antibodies, analyzed 48 h after the virus infection by flow cytometry. The CD34+CD38-HSPCs were gated out of total CD34+ cells (left panels) and analyzed for percent of GFP-expressing cells (right panels) after the virus infection or sham infection (uninfected cells, no virus). The GFP-expressing CD34+CD38-HSPCs cells are shifted upward long the Y-axis (GFP) and designated by a rectangle. Percent of GFP-positive cells after virus infection or in uninfected cells is shown on the corresponding dot plots. For this analysis, CD34+ cells were gated out of live and single cells, prior to analyzing percent GFP positive cells.

FIG. 18 shows the efficacy and selectivity of human hematopoietic cell transduction with AVID-388 vector in vivo after intravenous administration to humanized huCD34-NSG mice. Forty-eight hours after intravenous administration of AVID-388 at a dose of 8×10¹⁰ virus particles per mouse or Mock (control, buffer-injected group), mice were sacrificed, and bone marrow was harvested for flow cytometry analyses. Due to the extreme paucity of HSPCs, harvested bone marrow was pooled from 3 to 7 mice per group prior to CD34+ cell enrichment, staining with antibodies to different cell markers, and flow cytometry analysis of distribution of GFP+ cells among indicated bone marrow cell populations. The experiment was repeated 3 times, with mice from non-treated, Buffer-injected, and AVID-388-injected groups analyzed simultaneously in three batches. The results from three independent biological replicates were averaged and the average transduction efficacy of indicated primitive hematopoietic cell populations are shown above corresponding graph bars.

FIG. 19 shows in vivo transduction of primitive human CD34+CD49f+ hematopoietic cell populations in the bone marrow of humanized huCD34+ NSG mice after intravenous administration of AVID-388 at a dose of 8×10¹⁰ virus particles per mouse. Dot plots show gating parameters used for analysis of the proportion of GFP-expressing cells in human CD34+CD38-CD45RA-CD90+/−CD49f+ cell subsets in the bone marrow of mice 48 h after intravenous administration of AVID-388. The percent GFP-positive cells within indicated cell populations is shown.

FIG. 20A shows kinetics of growth of subcutaneous tumors, derived from human lung adenocarcinoma A549 cells, grafted into NSG mice after intravenous therapy with AVID-388 virus or Buffer (Control). N=7 for Control, buffer-treated cohort; N=9 for AVID-388-treated cohort.

FIG. 20B shows Log-rank survival plot of NSG mice with subcutaneous tumors, derived from human lung adenocarcinoma A549 cells, after therapy with buffer (Control) or AVID-388 virus. N=7 for Control, buffer-treated cohort; N=9 for AVID-388-treated cohort. Survival curves, corresponding to each treatment cohort, are indicated by arrows. P<0.0001. P value was calculated using GraphPad Prism 9.4.0 software.

FIG. 21A shows kinetics of growth of subcutaneous tumors, derived from human lung adenocarcinoma A549 cells, grafted into NSG mice after intravenous therapy with AVID-317 virus or Buffer (Control). N=7 for each treatment cohort.

FIG. 21B shows Log-rank survival plot of NSG mice with subcutaneous tumors, derived from human lung adenocarcinoma A549 cells, after therapy with buffer (Control) or AVID-317 virus. N=7 for each treatment cohort. Survival curves, corresponding to each treatment cohort, are indicated by arrows. P=0.0002. P value was calculated using GraphPad Prism 9.4.0 software.

FIG. 22 shows Log-rank survival plot of NCr-Nude mice with disseminated lung tumors, produced by human lung adenocarcinoma A549 cells, after intravenous administration of three doses of AVID-317 virus, injected at 5×10¹⁰ v.p. per mouse at 48 hours interval, or buffer (Control). Mice after therapy were sacrificed when they experienced a 20% weight loss against their weight prior to the beginning of treatment. N=11 for Buffer-treated cohort. N=9 for AVID-317-treated cohort. P=0.0029. Survival curves, corresponding to each treatment cohort, are indicated by arrows. P value was calculated using GraphPad Prism 9.4.0 software.

FIG. 23 shows the schematic diagram of the multiplexed targeted therapeutic adenovirus platform vector of the invention comprising mutated penton and fiber, which is able to mediate attachment to cell through binding to CD46 or DSG2, but not coxsackievirus and adenovirus receptor, CAR. The striped box indicates deletion of an RGD amino acid motif and insertion of a peptide into the penton base RGD loop, enabling virus entry into cells via non-RGD-interacting integrins.

### SUMMARY

The present application provides compositions and methods for increasing selectivity and efficacy of transduction of human cells, including human long-term repopulating and hematopoietic stem cells (LT-HSC), lineage-committed human cells of hematopoietic origin, human cells of non-hematopoietic origin, and human cancer cells, by adenovirus vectors through multiplexed targeting of virus attachment and internalization receptors. The multiplexed targeted adenovirus vectors of the invention attach to cells via mutated or native fibers comprising fiber knob domains that interact with CD46 or DSG2 receptors, but not with coxsackie-adenovirus receptor, and enter human cells via mutated pentons that have the RGD amino acid motif deleted, and that were further engineered to interact with non-RGD-binding integrin classes expressed on the surface of target cells.

Multiplexing targeting Ad vector receptor specificities through the combination of restricted fiber-specific attachment receptors, deletion of RGD amino acids from the penton base protein, and insertion into Ad penton base of peptides that lack the RGD amino acid motifs and enable vector interaction with integrin classes expressed on human cells, allows for the improvement of selectivity and efficacy of vector-mediated transduction of human cells, including LT-HSC cells, in vitro and after intravenous vector administration in vivo.

In a preferred embodiment of present application, the Ad vector, if based on human Ad serotype HAdv-C5, comprises mutated penton base with deleted RGD amino acid motifs and further modified by the substitution of the RGD loop of the HAdv-C5 penton base protein with a short artificial a6-integrin interacting peptide containing amino acids SIKVAV (SEQ ID NO:1).

In some embodiments, the a6-integrin-interacting peptide is inserted into the surface-exposed region of the penton base capsid protein from an Ad vector of a human or animal serotype, encoding mutated or a native fiber protein, comprising at least the fiber knob domain that is able to interact with CD46 as a cell attachment receptor. Such fiber knob domain or the native fiber may be derived from, but not limited to, human adenovirus serotypes HAdv-B11, HAdv-B16, HAdv-B21, HAdv-B34, HAdv-B35, and HAdv-B50.

In a more preferred embodiments, the 6-integrin-interacting peptide is inserted into the surface-exposed region of the penton base capsid protein from an Ad vector of a human or animal serotype, encoding mutated or a native fiber protein, comprising at least the fiber knob domain that is able to interact with DSG2 as a cell attachment receptor. Such fiber knob domain or the native fiber may be derived from, but not limited to, human adenovirus serotypes HAdv-B3. HAdv-B7, HAdv-B14, and HAdv-B55.

In some embodiments, the modified Ad vectors comprising the non-RGD-integrin interacting peptide and the fiber knob domain that binds to CD46 or DSG2 further comprise one or more optional mutations in the adenovirus hexon to reduce virus toxicity, avoid neutralization by humoral factors of innate and adaptive immunity, and improve in vivo virus pharmacokinetics and pharmacodynamics after intravenous administration.

In some embodiments, the modified Ad vectors of the present application can additionally or alternatively include peptide insertions to confer interactions with non-RGD-interacting integrin classes, other than a6-integrins, including non-RGD-interacting integrin classes expressed on the surface of human lymphocytes.

The Ad vectors of the invention can be used for in vitro and in vivo delivery of reporter genes, therapeutic genes, cytotoxic genes, genes expressing shRNA, anti-sense RNA, Linc-RNA, guide RNA, genes expressing genomic DNA- or RNA-editing enzymes, or any combination of these genes for gene therapy applications, where targeted expression of such genes may provide therapeutic benefits to patients with genetic or acquired diseases.

The invention also provides methods of administering an Ad of the invention with therapeutic intent to patients with genetic or acquired diseases seeking treatment, wherein the method comprises administering a patient seeking treatment with such a vector.

The invention also provides methods of delivering a gene to a non-hepatic mammalian cell with the Ads of the invention through contacting the host cell with the Ad in vivo.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### REFERENCE TO ELECTRONIC SEQUENCE LISTING

The application contained a Sequence Listing which has been submitted electronically in. XML format and is hereby incorporated by reference in its entirety. Said. XML copy, created Jul. 23, 2024, is names “2023-007 CIP.xml” and is 46,132 bytes in size. The sequence listing contained in this .XML file is part of the specification and is hereby incorporated by reference herein in its entirety.

### I. Definitions

Unless defined otherwise, all technical and scientific terms used herein have the same meaning as commonly understood by one of ordinary skill in the art to which this invention belongs. For purposes of the present application, the following terms are defined below.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

As used herein, the term “adenovirus vector” should be construed as additionally referring to an adenovirus particle or virion, unless the context clearly suggests otherwise. In addition, where the phrase stated “adenovirus vector comprising [a protein]”, the phrase should be construed as an adenovirus vector encoding the protein.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “homology” and “% identity” should be construed as being equivalent to “similarity”. Thus, for example, if the use of the word homology is used between two non-natural sequences, this should not be construed as necessarily indicating an evolutionary relationship between these two sequences, but rather the similarity or relatedness between the two sequences. Many of the methods for determining homology between two evolutionarily related molecules are routinely applied to any two or more nucleic acids or proteins for the purpose of measuring sequence similarity regardless of whether they are evolutionarily related or not.

In general, one way to define any known variants and derivatives or those that might arise from the disclosed genes and proteins herein is through defining the variants and derivatives in terms of homology to specific known sequences. Variants of the genes, proteins, and peptides disclosed herein typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology (or identity) to the stated sequence or the native sequence. Those of skill in the art can readily determine the homology of two nucleic acid or amino acid sequences. For example, the homology can be calculated after aligning the two sequences so that the homology is at its highest level. It should be understood that any of the methods typically used can in certain instances produce slightly different results. Consequently, to the extent that a % identity is determined from one of these methods, the sequences would be said to have the stated identity resulting therefrom.

In this specification and in the claims which follow, reference will be made to a number of terms which shall be defined to have the following meanings: “Optional” and “optionally” mean that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### II. Compositions of the Present Application

Disclosed are structural components for preparing and using the compositions of the present application, as well as the compositions themselves to be used within the methods disclosed herein. It should be understood that where combinations, subsets, interactions, groups, etc. of these elements are disclosed, where specific reference to various individual and collective combinations and permutations of these compounds may not be explicitly disclosed, each is specifically contemplated and described herein.

In one aspect, the present invention provides for novel adenovirus (Ad) vectors with modified capsid proteins. Adenovirus is a ubiquitous pathogen causing a wide range of human diseases, which include respiratory tract infections, conjunctivitis, hemorrhagic cystitis, and gastrointestinal diseases. In immunocompetent patients, Ad infections are self-limited, and after resolution of the acute infection, the virus remains latent in lymphoid and renal tissues. In contrast, in immunocompromised patients Ad infections may cause life threatening or even fatal fulminant hepatitis and disseminated infection of other tissues.

There are currently over 60 characterized human Ad serotypes which are divided into seven “species” (formally subgroups) from A to G. Although various Ad serotypes may initiate infections via different transmission routes, and utilize distinct virus attachment receptors, the host factors and cell types controlling tissue specificity of Ad infection in vivo remain insufficiently understood.

The Ad infectious cycle occurs in two steps. The early phase precedes the initiation of replication and makes it possible to produce the early proteins regulating the replication and transcription of the viral DNA. The replication of the genome is followed by the late phase during which the structural proteins that constitute the viral particles are synthesized. The assembly of the new virions takes place in the host cell nucleus. In a first stage, the viral proteins assemble to form empty capsids of icosahedral structure into which the genome is encapsidated. The assembled virus includes a penton base and fiber. The Ads liberated from cells are then capable of infecting other permissive cells. The fiber and the penton base proteins present at the surface of the capsids play a role in the cellular attachment of the virions and their internalization.

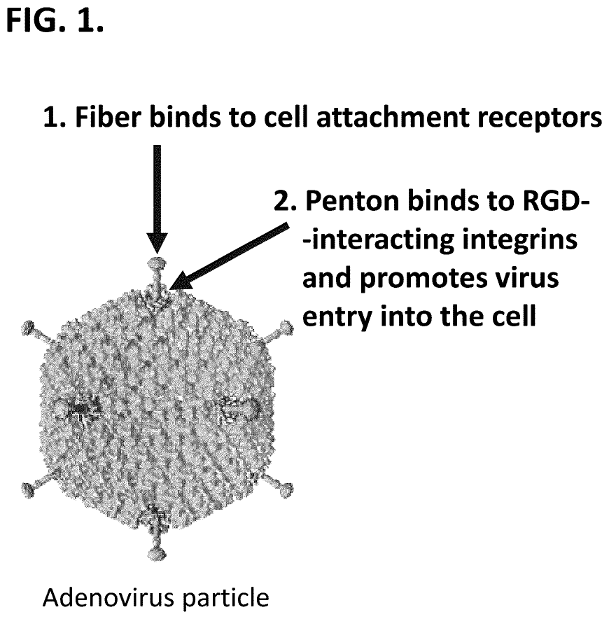

In vitro studies have demonstrated that Ad infection starts with the virus binding to a high affinity primary attachment receptor on the cell surface. The trimeric Ad fiber protein mediates this interaction when its distal knob domain binds to a specific cellular receptor (FIG. 1). For binding to cells, species A, C, D, E, and F human Ads may utilize the coxsackievirus and Ad receptor (CAR); however, human species B Ad utilize CD46 or DSG2 as a high affinity cellular attachment receptor. Fiber-mediated binding of Ad to cells is followed by RGD amino acid motif-mediated binding of the viral penton base protein to cellular integrins of RGD-interacting classes, primarily but not limited to αvβ3 and αvβ5 classes (FIG. 1). This interaction induces integrin activation and cytoskeleton rearrangement that facilitates internalization of the virus particle into the cell.

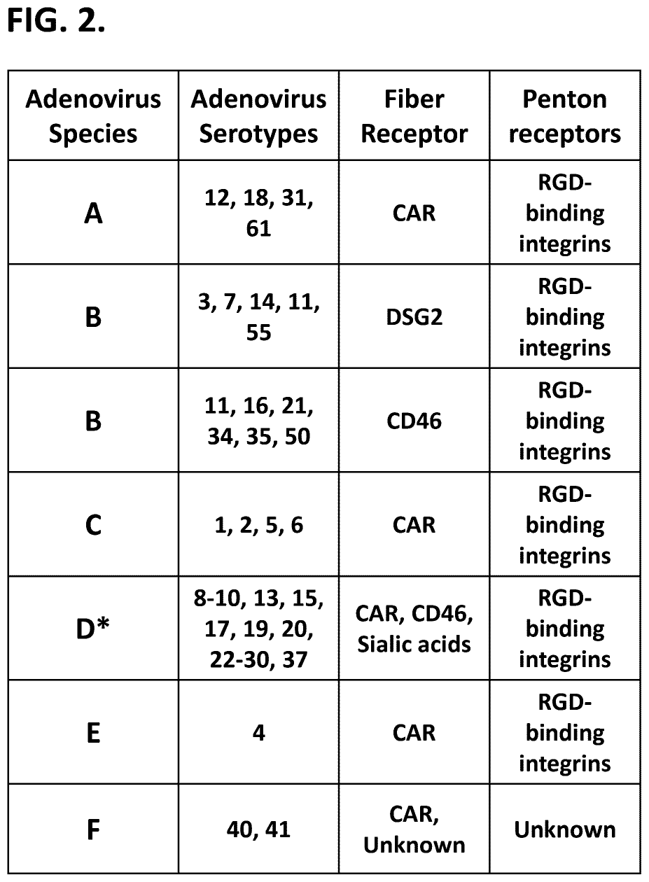

Although human Ads of different serotypes utilize different cell attachment receptors (FIG. 2), all adenovirus serotypes except for human species F adenoviruses HAdv-F40 and HAdv-F41, comprise RGD amino acid motif in the penton base protein and therefore can utilize RGD-interacting integrin classes for internalization into cells (FIG. 2).

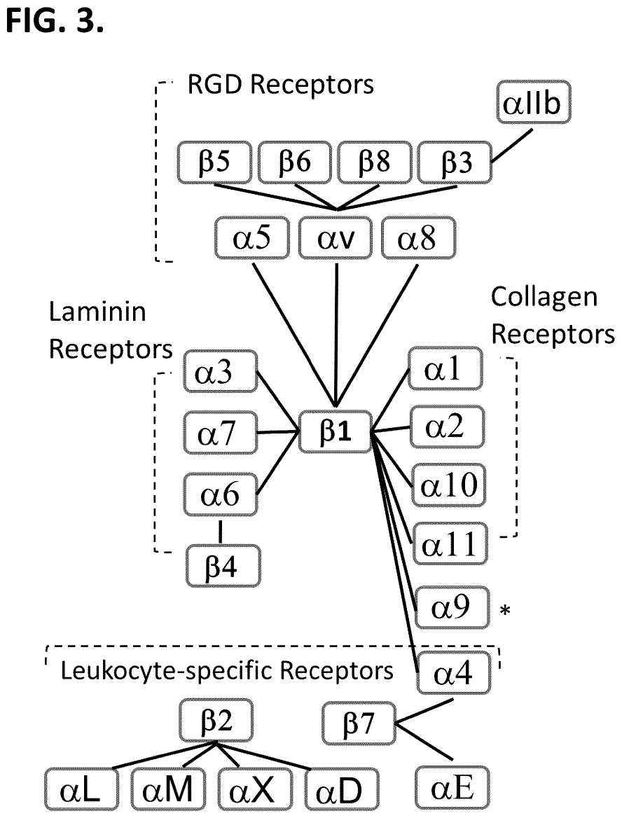

Integrins are transmembrane cellular proteins that are localized at the cell surface and function as heterodimers, which are formed by the a- and b-subunits. Integrins are classified based on their ligand specificity and expression on leukocytes (FIG. 3). The RGD-interacting, “RGD-receptors” integrin classes is the largest class of integrins broadly expressed on all cell types and include a5b1, avb1, a8b1, avb3, avb5, avb6, avb8 and aIIbb3 (FIG. 3). The “Laminin receptors” integrin classes include a3b1, a6b1, a7b1, and a6b4 (FIG. 3). The “Collagen receptors” integrin classes include a1b1, a2b1, a10b1, a11b1. The integrin class of “Leukocyte receptors” are expressed primarily on hematopoietic cells and include aLb2, aMb2, aXb2, and aDb2 integrins, as well as a4b1, a4b7, and aEb7 integrins. Integrins of a9b1 class are expressed on variety of cells and interact with variety of ligand-receptors, including a-disintegrin and metalloprotease (ADAM) family, clastic microfibril interface-located protein1 (EMILIN1), vascular endothelial growth factor (VEGF), the extra domain A (EDA) of fibronectin, tenascin-C (TNC), osteopontin (OPN), vascular cell adhesion molecule-1 (VCAM-1) and C-motif-ligand-1 (XCL1)/lymphotactin.

The type of integrins expressed on a particular cell type defines its ability to traffic through the body to or reside in the appropriate niches within specific tissues where cells perform their functions. Although certain selectivity exists in integrin classes that are expressed on any particular cell type, each specific cell type expresses a variety of integrin classes to support diverse cellular functions and ability of cell attachment to various extracellular matrixes in different tissue microenvironments and physiological or pathological contexts. For instance, human airway epithelial cells express eight different integrin heterodimers, including a2b1, a3b1, a6b4, a9b1, a5b1, avb5, avb6, avb8. Human NK cells circulating in the blood express αLβ2, αMβ2, α4β1, α5β1, and α6β1 integrins. Human melanoma tumor cells express a4b1, a2b1, and avb3 integrin classes, which drive differential migration and homing of tumor cells to distant organs, e.g. brain, liver, lung, bone tissue, and lymph nodes.

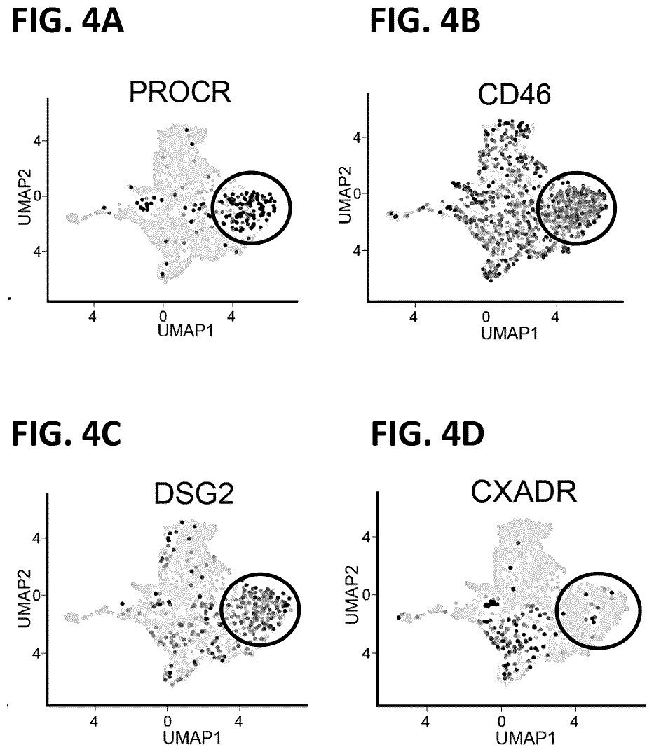

The genome-wide transcriptional profiling at a single cell level, using single cell RNA sequencing, allows for a determination of expression of any gene of interest and complexity among early hematopoietic progenitor cell populations using UMAP plots (as reported in (Ranzoni et al., 2021)). Using single cell transcriptomics data, reported by (Ranzoni et al, 2021), one can visualize the distribution of cells, expressing any particular gene of interest on the UMAP plots revealing how broadly a particular gene is expressed in the analyzed cell pool or in a defined cell population. Specifically, visualization of cells expressing human PROCR gene allows to determine localization of human LT-HSC cells among other human hematopoietic progenitor cell populations (FIG. 4A, highlighted by a circle).

Analysis of expression of adenovirus attachment and internalization receptors on human LT-HSCs showed that adenovirus attachment receptors CD46 and DSG2 are highly expressed in LT-HSCs (FIG. 4B and FIG. 4C). In contrast, the adenovirus receptor CXADR (CAR) is not expressed in LT-HSC cells (FIG. 4D).

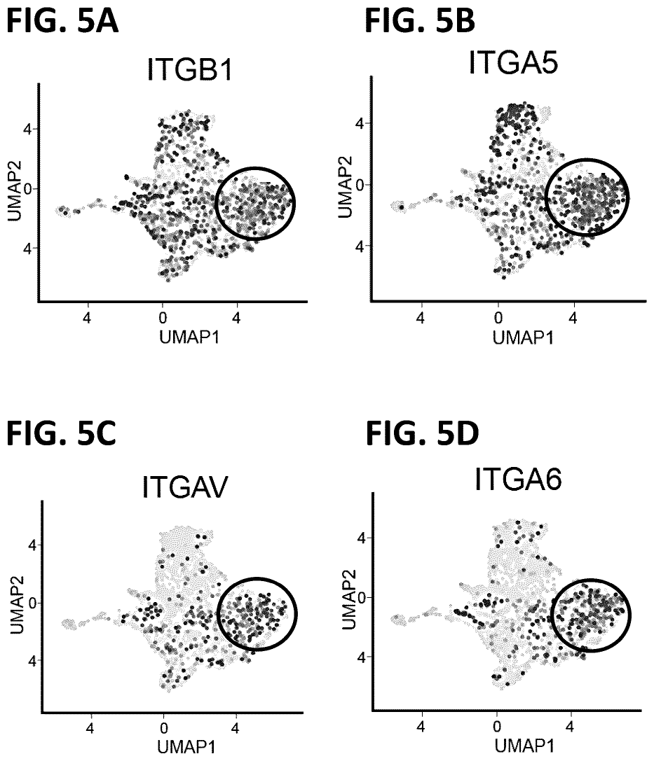

As shown in FIG. 5, integrin genes that are highly expressed in LT-HSC cells include gene encoding for the integrin b1 (encoded by ITGBI gene) classes (FIG. 5A). These integrins dimerize with the majority of a-integrin classes to form functional integrin heterodimer receptors at the cell surface (FIG. 3). Among integrins of a-classes that are highly expressed in LT-HSC cells, there are two RGD-interacting integrins, namely a5 and av (encoded by ITGA5 and ITGAV genes, respectively; see FIG. 5B and FIG. 5C). and integrins that do not interact with RGD-motif-containing ligands, namely a6 integrin classes, encoded by ITGA6 (FIG. 5D).

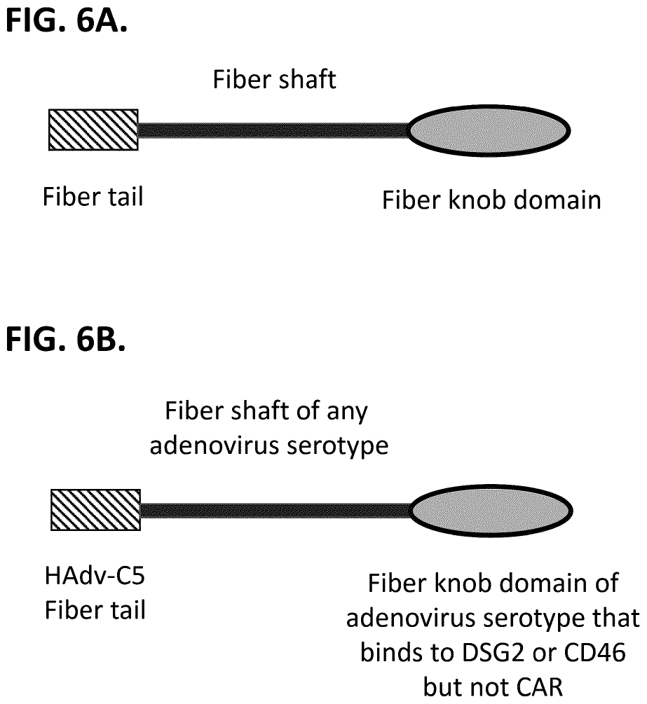

The Ad vectors of the present application comprise fiber protein that is able to utilize CD46 or DSG2 receptors, but not CAR, to mediate virus attachment to mammalian cells. Ad vectors of the invention comprise fiber proteins of the general structure shown in FIG. 6A, particularly the structure shown in FIG. 6B. In one non-limiting embodiment, a fiber structure suitable for mammalian cell transduction is based on the HAdv-C5 serotype encoding the fiber tail domain in FIG. 6B, specifically the fiber tail domain portion present in SEQ ID NO:2.

In some embodiments, the fiber shaft domain may be derived from any serotype and containing a fiber knob domain binding to the CD46 or DSG2 cell attachment receptors as shown in FIG. 6B. A person of ordinary skill in the art can recognize amino acid sequences encoding fiber shaft, tail, and knob domains within the entire amino acid sequence of the fiber protein based on the publicly available structural information for adenovirus fibers of different serotypes.

Non-limiting examples of adenovirus fibers recognizing the CD46 cell attachment receptor include fibers naturally occurring in human adenoviruses of the following serotypes: HAdv-B11 (SEQ ID NO:3), HAdv-B16 (SEQ ID NO:4), HAdv-B21 (SEQ ID NO:5), HAdv-B34 (SEQ ID NO:6), HAdv-B-35 (SEQ ID NO:7), and HAdv-B50 (SEQ ID NO:8).

Non-limiting examples of adenovirus fibers binding the DSG2 cell attachment receptor include fibers naturally occurring in human adenoviruses of the following serotypes: HAdv-B3 (SEQ ID NO:9), HAdv-B7 (SEQ ID NO: 10), HAdv-B14 (SEQ ID NO:11), and HAdv-B55 (SEQ ID NO:12).

A non-limiting example of a mutated, receptor-re-targeted, chimeric adenovirus fiber binding the CD46 cell attachment receptor and containing a fiber tail domain from the human HAdv-C5 serotype includes a chimeric fiber protein comprising fiber knob domain derived from human adenovirus serotype HAdv-B35 and comprising the amino acid sequence of SEQ ID NO: 13.

A non-limiting example of a mutated, receptor-re-targeted, chimeric adenovirus fiber binding the DSG2 cell attachment receptor and containing a fiber tail domain from the human HAdv-C5 serotype includes a chimeric fiber protein comprising fiber knob domain derived from the human adenovirus serotype HAdv-B14 and comprising the amino acid sequence of SEQ ID NO: 14.

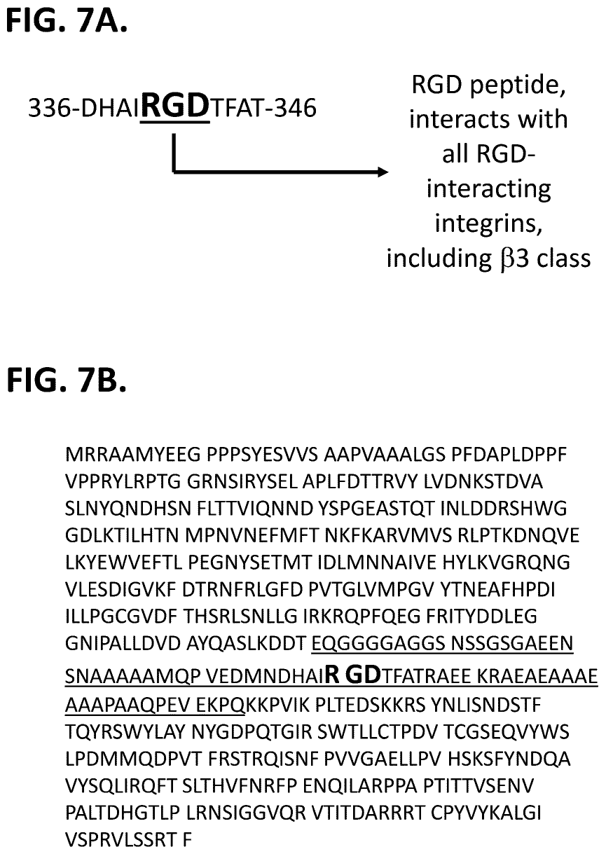

The adenovirus penton RGD loop comprises the R-G-D amino acid motif of SEQ ID NO: 15, which is present in the penton base protein of human adenovirus serotype HAdv-C5 (FIG. 7A, SEQ ID NO:16 and SEQ ID NO:17). The RGD amino acid motif in the penton base mediates virus binding to RGD-interacting integrin classes, including b3 integrins expressed on resident macrophages in liver and spleen, which trigger b3 integrin-dependent production of inflammatory cytokines and chemokine upon virus entry into these phagocytic cells. In human adenovirus HAdv-C5 serotype, the RGD loop is large (FIG. 7B, and SEQ ID NO: 17), compared to human adenovirus serotypes of other species.

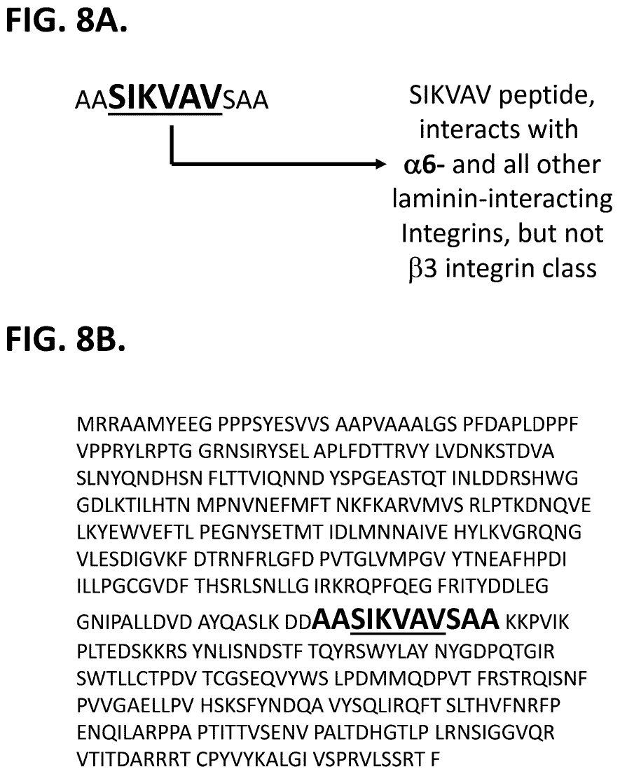

The preferred embodiments of the invention are Ad vectors comprising the penton RGD amino acid motif deletion and substitution. The non-limiting example of such deletion and substitution is the insertion of an artificial peptide of SEQ ID NO: 18 comprising SIKVAV amino acid motif (SEQ ID NO:1, FIG. 8A) in place of the RGD loop of the penton (SEQ ID NO: 19 and FIG. 8B). In another non-limiting example, the non-RGD motif-containing peptide SIKVAV (SEQ ID NO: 1) can be inserted into and in place of the mutated RGD loop of the penton (SEQ ID NO: 20).

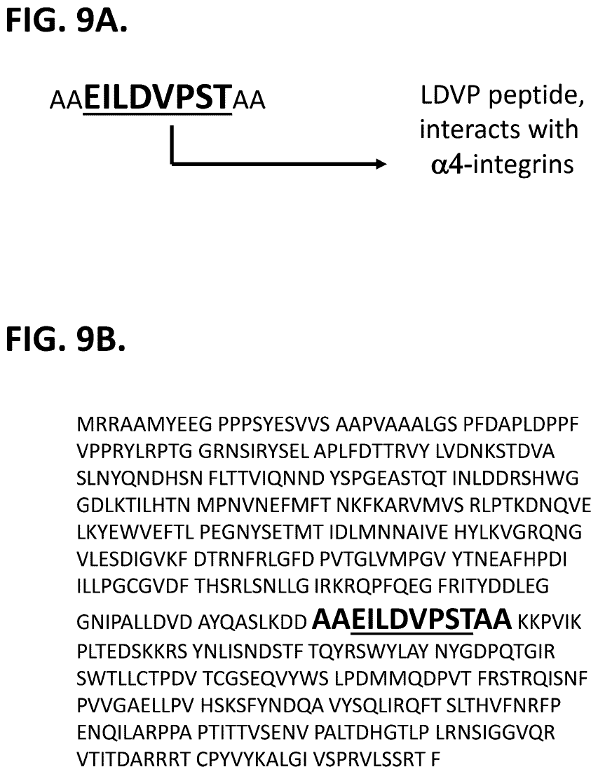

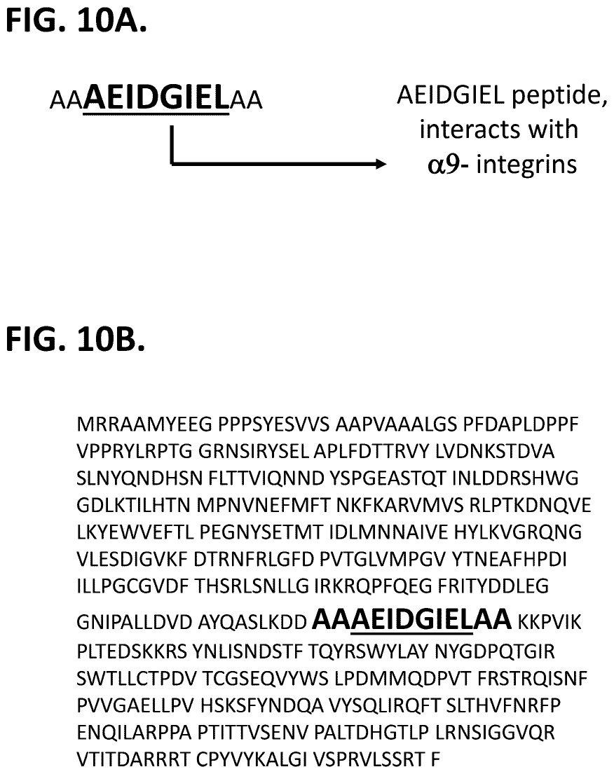

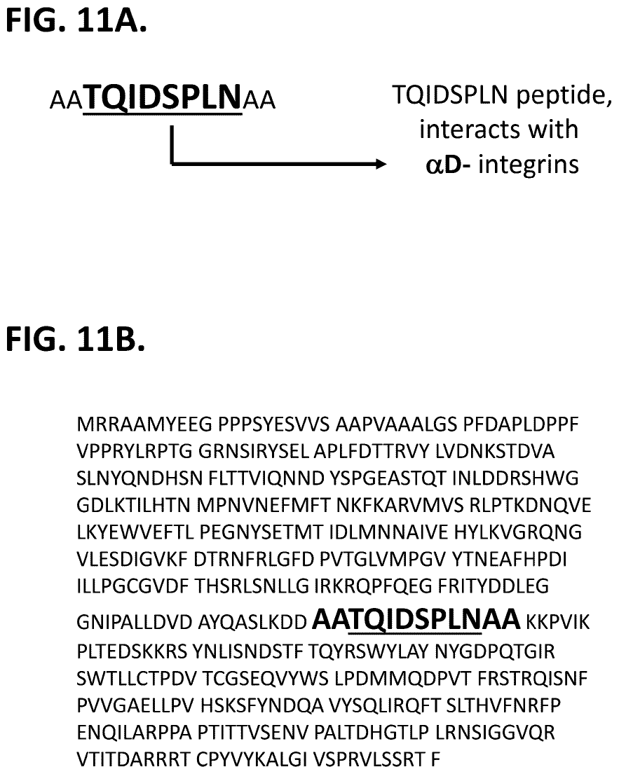

In some embodiments, Ad vectors of the present application include any one of the Ad vectors encoding a modified penton base protein comprising a non-RGD-containing peptide that can mediate virus entry into target cells of interest via receptors expressed on the target cells that may include, without limitation, a4-, a9-, or aD-integrins and further comprise sequences encoding fiber knob domains that are able to utilize CD46 or DSG2, but not CAR, for vector attachment to cells. The non-limiting examples of non-RGD-containing peptides and mutated penton base proteins, comprising such peptides are shown in FIG. 9 (SEQ ID NO:21 and SEQ ID NO: 22 for peptide, interacting with a4-integrin classes), FIG. 10 (SEQ ID NO:23 and SEQ ID NO: 24 for peptide, interacting with a9-integrin classes), and FIG. 11 (SEQ ID NO:25 and SEQ ID NO: 26 for peptide, interacting with aD-integrin classes).

In some embodiments, an Ad vector comprising mutated fiber and mutated penton may be based on any human or animal serotype, other than HAdv-C5, which comprises a native or mutated fiber protein allowing for vector to utilize CD46 or DSG2, but not CAR, as a cell attachment receptor.

As noted above, the Ad vectors described herein can be based on human Ad serotype 5 (HAdv-C5). However, the Ad vectors can be based on any other human or animal Ad serotype. For example, non-human Ads can include, without limitation, canine, avian, bovine, murine, ovine, porcine, or simian origin. It is understood that if the Ad vector is based on non-human or non-HAdv-C5 serotype, where the natural fiber structure allows for virus binding to CD46 or DSG2, but not CAR, the preferred embodiment of the invention based on such non-human or non-HAdv-C5 serotype should comprise mutated penton base protein with deleted RGD amino acid motif and insertion of a non-RGD-containing peptide in place of the RGD loop of the penton base, allowing for selective and efficient infection of mammalian cells needing therapy, as disclosed herein.

In certain preferred embodiments, the Ad of the present application can be a recombinant and replication-defective Ad (i.e., incapable of autonomously replicating in a host cell). Such a replication-deficient Ad can include, for example, a mutation or deletion of one or more viral regions, such as, for example, all or part of the E1 region and/or E4 region. The genome of an Ad optionally can include additional deletions or mutations affecting other regions, such as, for example, the E2, E3 and/or L1-L5 regions, including complete deletion of the virus coding sequences and replacement with non-Ad DNA (so called “helper-dependent” vectors).

In other preferred embodiments, the Ad vector of the present application can be replication-competent or replication-restricted and engineered to replicate specifically in cancer cells.

An Ad vector of the present application can include one or more genes of interest contained within a nucleic acid segment that is introduced into an Ad vector. The genes of interest can be placed under the control of the elements necessary for their expression in a specific host cell. The gene of interest is typically a human or non-human heterologous gene (i.e., a non-Ad gene). The gene of interest can be, for example, genomic, cDNA (complementary DNA), a hybrid or chimeric gene (e.g., a minigene lacking one or more introns), or the like. It can be obtained, for example, by conventional molecular biology techniques and/or by chemical synthesis. A gene of interest can encode, for example, an antisense RNA, shRNA, lncRNA, or siRNA, guide-RNA, a ribozyme, or an mRNA that can be translated into a polypeptide of interest. Polypeptides of interest include e.g., nuclear, cytoplasmic, membrane, secreted or other types of proteins, including proteins that can perform targeted editing of DNA sequences in human genomic DNA. Further, the polypeptide of interest can be, for example, a polypeptide as found in nature, a chimeric polypeptide obtained from the fusion of heterologous sequences of diverse origins, or of a polypeptide mutated relative to the native sequence having improved and/or modified biological properties.

In certain embodiments, the Ad vector can include a gene of interest that is configured to achieve a predetermined function or outcome. The gene of interest can encode, for example and without limitation, cytokines or lymphokines (α-, β- or γ-interferon, interleukins (e.g., IL-1α, IL-2, IL-6, IL-10, IL-12, IL-15, IL-15R, IL-24, and alike)), colony stimulating factors (e.g., GM-CSF, C-CSF, M-CSF, or the like); cellular or nuclear receptors, including those recognized by pathogenic organisms (e.g., viruses, bacteria or parasites); proteins involved in activation of innate immune signaling of prokaryotic or eukaryotic origin (e.g., bacterial flagellin, or the like); proteins involved in triggering a genetic diseases (e.g., factor VII, factor VIII, factor IX, dystrophin or minidystrophin, insulin, CFTR protein (Cystic Fibrosis Transmembrane Conductance Regulator)); growth hormones (e.g., insulin, hGH or the like); metabolic or homeostatic enzymes (e.g., urease, renin, thrombin, or the like); genomic DNA-editing or host genomic DNA modifying enzymes, RNA-editing enzymes (e.g., CRISPR-Cas enzymes and enzymes with similar functions), enzyme inhibitors (e.g., α1-antitrypsin, antithrombin III, viral protease inhibitors, or the like); polypeptides with antitumor effect (e.g., which are capable of at least partially inhibiting the initiation or the progression of tumors or cancers), such as antibodies, inhibitors acting on cell division or transduction signals, products of expression of tumor suppressor genes (specifically, but without limitation, p53 or pRb), cell adhesion molecules, proteins stimulating the immune system, or the like); proteins of the class I or II major histocompatibility complex or regulatory proteins acting on the expression of the corresponding genes; polypeptides capable of inhibiting a viral, bacterial or parasitic infection or its development (e.g., antigenic polypeptides having immunogenic properties, antigenic epitopes, antibodies, transdominant variants capable of inhibiting the action of a native protein by competition, or the like); toxins (e.g., herpes simplex virus 1 thymidine kinase (HSV-1-TK), ricin, cholera toxin, diphtheria toxin, or the like) or immunotoxins; markers (β-galactosidase, luciferase, Green Fluorescent Protein, or the like); polypeptides having an effect on apoptosis (e.g., inducer of apoptosis: Bax, or the like, blocker of apoptosis Bcl2, Bcl-x, or the like); cytostatic agents (e.g., p21, p16, Rb, or the like); apolipoproteins (e.g., apoE or the like); superoxide dismutase, catalase, nitric oxide synthase (NOS); growth factors (e.g., Fibroblast Growth Factor (FGF), Vascular Endothelial Cell Growth Factors (VEGFs), insulin, or the like), or others genes of therapeutic, prophylactic, or research interest. It should be noted that this list is not limiting and that other genes can also be used.

The Ad optionally can include a selectable gene which allows for selection or identification of the infected cells. Suitable selectable genes include, for example, Neo (neomycin phosphotransferase), DHFR (Dihydrofolate Reductase), CAT (Chloramphenicol Acetyltransferase), PAC (Puromycin Acetyltransferase), GPT (Xanthine-Guanine Phosphoribosyltransferase), MGMT (O6-methylguanine-DNA methyltransferase), and the like.

In certain embodiments, the gene of interest can further include elements necessary for the selective or regulated expression of the gene in a select host cell. Such elements include, for example, elements facilitating transcription of the gene into RNA and/or the translation of an mRNA into a protein and include promoter, enhancers, and insulators. Suitable promoters include, for example, those of eukaryotic or viral origin. Suitable promoters can be constitutive or regulatable (e.g., inducible). A promoter can be modified to increase promoter activity, suppress transcription-inhibiting regions, make a constitutive promoter regulatable, introduce a restriction site, and the like. The non-limiting examples of suitable promoters include, for example, the cytomegalovirus (CMV) promoter, Rous Sarcoma Virus (RSV) promoter, HSV-1 TK promoter, Simian Virus 40 (SV40) early promoter, Adenovirus (Ad) major late promoter (MLP), murine or human promoters from phosphoglycerate kinase (PGK), metallothionein (MT), the liver-specific al-antitrypsin and albumin promoters, lymphocyte-specific immunoglobulin promoters, endothelial-specific vascular endothelial growth factor receptor 1 (VEGFR-1) promoter, tumor-specific a-fetoprotein (AFP), MLJC-1, and prostate specific antigen (PSA) promoters, and endothelial cell specific flt promoter.

A gene of interest can also include additional elements for the expression (e.g., an intron sequence, a signal sequence, a nuclear localization sequence, a transcription termination sequence, a site for initiation of translation of the IRES type, or the like), for its maintenance in the host cell etc.

Further provided herein is a host cell, infected with an Ad according to the present disclosure or capable of being obtained by a method according to the present application.

### III. Pharmaceutical Compositions

Another aspect of the present application relates to a pharmaceutical composition that comprises an adenovirus vector or polynucleotide of the present application and a pharmaceutically acceptable carrier.

The pharmaceutical compositions are co-administered with at least one pharmaceutically acceptable carrier for in vivo or ex vivo delivery. As used herein, the term “pharmaceutically acceptable” is used with reference to a material that is not biologically or otherwise undesirable, e.g., the material may be administered to a subject, along with the Ad vector or cells, without causing any undesirable biological effects or interactions in a deleterious manner with any of the other components of the pharmaceutical composition in which it is contained. In some embodiments, the carrier can be selected to minimize degradation of the active components and minimize any adverse side effects in the subject, as would be well known to one of ordinary skill in the art.

The compositions may be administered parenterally (e.g., intravenously), orally, by intramuscular injection, by intraperitoneal injection, transdermally, extracorporeally, topically, or by inhalation through the mouth or nose. The exact amount of the composition required will vary from subject to subject, depending on the species, age, weight and general condition of the subject, the severity of the allergic disorder being treated, the particular nucleic acid or vector used, its mode of administration and the like. An appropriate amount can be determined by one of ordinary skill in the art.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

The pharmaceutical composition may be formulated in solution, suspension, or incorporated into microparticles, liposomes, or cells, including within human LT-HSC or tumor cells following their in vitro transduction with the viral vectors of the present application and subsequently used for administration to patients with therapeutic intent.

### IV. Therapeutic Uses

In one aspect, disclosed herein are methods of treatment according to which a therapeutically effective quantity of an Ad according to the present description or of a host cell is administered to a patient requiring such a treatment. Such methods can comprise treating a host with one or more pharmaceutical entities prior to, during, or after Ad administration.

The adenovirus vectors of the invention can be used as a gene delivery platform for in vitro, ex vivo, and in vivo delivery of reporter genes, therapeutic genes, cytotoxic genes, genes expressing shRNA, anti-sense RNA, Linc-RNA, guide-RNA, genes expressing genomic DNA- or RNA-editing enzymes, or any combination of these genes for gene therapy applications in vitro, ex vivo, or in vivo, specifically after intravenous vector administration, where expression of such genes may provide therapeutic benefits to patients with genetic or acquired diseases.

The disclosed Ad compositions can be also used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the treatment of precancer conditions such as cervical and anal dysplasias, other dysplasias, severe dysplasias, hyperplasias, atypical hyperplasias, and neoplasias.

### EXAMPLES

### Example 1. Assembly and Analysis of the Therapeutic Platform Vector A VID-317, Comprising Mutated Penton Base Protein with Deletion of the RGD Motif and Comprising a Peptide Interacting with a6-Integrins, and Further Comprising Mutated, Chimeric, Re-Targeted Fiber that Interacts with CD46 Attachment Receptor

As a base for engineering AVID-317 vector, we used known in the art vector Ad5-GFP that is based on human adenovirus serotype HAdv-C5 and that comprises a deletion in the E3 region of the virus genome but otherwise comprises wild type sequences for all open reading frames encoding viral capsid proteins. We used adenovirus cloning techniques known in the art and replaced an open reading frame encoding the wild type penton base protein of SEQ ID NO: 17 with an open reading frame of penton of SEQ ID NO:20, comprising SIKVAV amino acid motif (SEQ ID NO: 1) in place of the RGD loop as shown in FIG. 8A. To enable efficient infection of human cells via CD46 receptor, we replaced an open reading frame encoding the wild type fiber of SEQ ID NO:2 with an open reading frame encoding mutated, receptor-re-targeted, chimeric Ad5/35 fiber (SEQ ID NO:13) that has HAdv-B35-derived fiber knob domain that binds to CD46 as a cell attachment receptor, fiber shaft domain derived from the fiber protein of a human adenovirus serotype HAdv-E4 (SEQ ID NO: 27), and a fiber tail domain from human adenovirus HAdv-C5. The AVID-317 virus was rescued from cloned genomic DNA following transfection in 293 cells. The AVID-317 virus was serially passaged on 293 cells and amplified up to a batch of over 5×15 cm dishes, and then, virus particles were isolated using standard cesium chloride gradient centrifugation protocols. The amount of virus particles produced for the AVID-317 vector was assessed by measuring concentration of genomic viral DNA in purified vector stock using spectrophotometry. 1 U of OD₂₆₀ was considered 1×10¹³ virus particles per ml. For gene delivery studies, a CMV-GFP gene expression cassette, comprising green fluorescent protein (GFP) reporter gene under the control of the CMV early promoter, was placed into the E3 region of AVID-317 genome. For anti-tumor activity studies, the E3 region of AVID-317 comprised adenovirus death protein (ADP) in the E3 region of the viral genome.

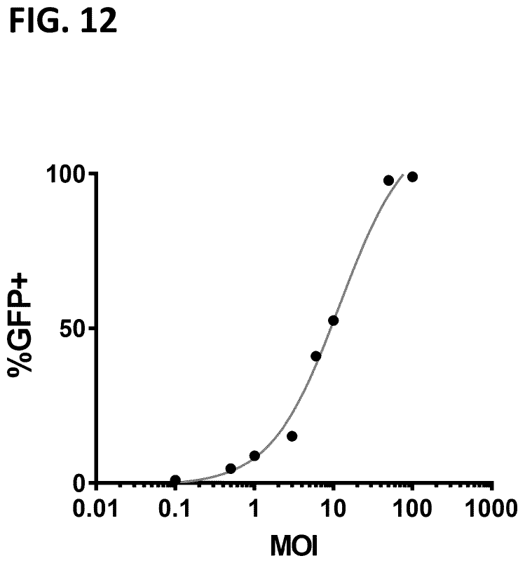

It is well established in the art that introduction of mutations in structural proteins of adenovirus and other gene transfer vectors can negatively affect the efficacy of cell infection by a mutated virus. To determine if adenovirus vector AVID-317, comprising mutations in penton and fiber, retains infectivity toward human cells, we infected human lung adenocarcinoma cell line A549 with Ad vector at various multiplicity of infection (MOIs) and analyzed cell transduction using flow cytometry methodology. Human lung adenocarcinoma A549 cells were seeded at a density of 1.5×10⁵ cells per well of the tissue-culture-treated 12 well plate one day prior to infection. 18-24 hours after seeding, cells were infected with AVID-317-CMV-GFP virus in DMEM+10% FBS media at MOIs ranging from 0.01 to 100 virus particles per cell. Percent of cells expressing GFP was analyzed 24 h later by flow cytometry using CytoFlex flow cytometer. The dose response curve of percent of GFP-positive cells versus virus dose was plotted and the EC50 dose was calculated using GraphPad Prism software. This analysis showed (FIG. 12) that EC50 for A VID-317-CMV-GFP virus on A549 cells is 11.91 virus genomes. The biological activity of 1 GFP transducing unit for A VID-317-CMV-GFP on A549 cells is equal to 12 v.g. per cell. This number indicates that the biological activity of the capsid-mutated virus with AVID-317 capsid configuration is well within the range of 5-20 v.g. per transduced cell, known in the art for vectors with wild type, unmodified capsids. This analysis demonstrated that mutations introduced in the vector of the invention have not negatively affected virus infectivity toward human cells.

### Example 2. Assembly and Analysis of the Therapeutic Platform Vector AVID-388, Comprising Mutated Penton Base Protein with Deletion of the RGD Motif and Comprising a Peptide Interacting with a6-Integrins, and Further Comprising Mutated, Chimeric, Re-Targeted Fiber that Interacts with DSG2 Attachment Receptor

As a base for engineering AVID-388 vector, we used known in the art vector Ad5-GFP that is based on human adenovirus serotype HAdv-C5 and that comprises a deletion in the E3 region of the virus genome but otherwise comprises wild type sequences for all open reading frames encoding viral capsid proteins. We used adenovirus cloning techniques known in the art and replaced an open reading frame encoding the wild-type penton base protein of SEQ ID NO: 17 with an open reading frame of penton of SEQ ID NO: 19, comprising SIKVAV amino acid motif (SEQ ID NO: 1) in place of the RGD loop as shown in FIG. 8. To enable efficient infection of human cells via DSG2 receptor, we replaced an open reading frame encoding the wild type fiber of SEQ ID NO:2 with an open reading frame encoding mutated, receptor-re-targeted, chimeric Ad5/14 fiber (SEQ ID NO:14) that has HAdv-B14-derived fiber knob domain that binds to DSG2 as a cell attachment receptor, fiber shaft domain derived from the fiber protein of a human adenovirus serotype HAdv-E4 (SEQ ID NO: 27), and a fiber tail domain from human adenovirus HAdv-C5. The AVID-388 virus was rescued from cloned genomic DNA following transfection in 293 cells. The AVID-388 virus was serially passaged on 293 cells and amplified up to a batch of over 5×15 cm dishes, and then, virus particles were isolated using standard cesium chloride gradient centrifugation protocols. The amount of virus particles produced for the AVID-388 vector was assessed by measuring concentration of genomic viral DNA in purified vector stock using spectrophotometry. 1 U of OD₂₆₀ was considered 1×10¹³ virus particles per ml. For gene delivery studies, a CMV-GFP gene expression cassette, comprising green fluorescent protein (GFP) reporter gene under the control of the CMV early promoter, was placed into the E3 region of the AVID-388 genome. For anti-tumor activity studies, the E3 region of AVID-388 comprised adenovirus death protein (ADP) in the E3 region of the viral genome.

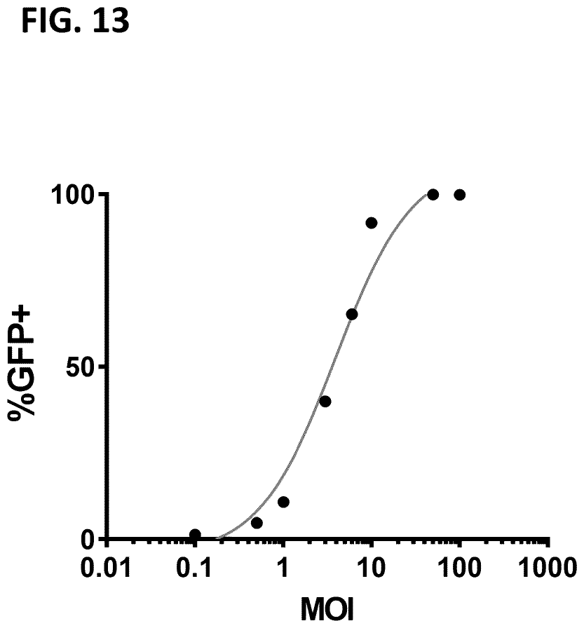

To determine if adenovirus vector AVID-388, comprising mutations in penton and a DSG2-interacting fiber, retains the infectivity toward human cells, we infected human lung adenocarcinoma cell line A549 with Ad vector at various multiplicity of infection (MOIs) and analyzed cell transduction using flow cytometry methodology. Human lung adenocarcinoma A549 cells were seeded at a density of 1.5×10⁵ cells per well of the tissue-culture-treated 12 well plate one day prior to infection. 18-24 hours after seeding, cells were infected with AVID-388-CMV-GFP virus in DMEM+10% FBS media at MOIs ranging from 0.01 to 100 virus particles per cell. Percent of cells expressing GFP was analyzed 24 h later by flow cytometry using CytoFlex flow cytometer. The dose response curve of percent of GFP-positive cells versus virus dose was plotted and the EC50 dose was calculated using GraphPad Prism software. This analysis showed (FIG. 13) that EC50 for AVID-388-CMV-GFP virus on A549 cells is 3.9 virus genomes. The biological activity of 1 GFP transducing unit for AVID-388-CMV-GFP vector on A549 cells is equal to 6.8 v.g. per cell. This number indicates that the biological activity of this DSG2-interacting AVID-388 vector platform is well within the range of 5-20 v.g. per transduced cell, known in the art for vectors with wild type, unmodified capsids. This analysis demonstrated that mutations introduced in the vector of the invention have not negatively affected virus infectivity toward human cells.

### Example 3. The Penton-Mutated CD46-Interacting and DSG2-Interacting Therapeutic Platform Vectors Trigger Significantly Reduced Production of Inflammatory Cytokines after Intravenous Administration, Compared to Unmodified Virus HAdv-B11

To analyze whether mutations introduced into novel AVID-317 and AVID-388 vectors may have adversely and unpredictably affected its safety after intravenous administration, as it was earlier reported in the art for Ad5: Ad48 vector with mutated hexon, we analyzed the amounts of inflammatory cytokines IL-6, TNF-α, IL-1b and IFN-g in plasma of mice 6 hours after the intravenous vector administration. For this analysis, 10-14-week-old wild type C57BL6 mice, females, with an average weight of 20 g were used. Mice were administered intravenously with an injection buffer (Control group), human adenovirus of HAdv-B11 serotype with unmodified wild type capsid, or novel AVID-317 and AVID-388 vectors at a very high dose of 1×10¹¹ virus particles per mouse. Virus samples were diluted in the injection buffer with the following composition: 150 mM NaCl, 10 mM Tris-HCL, pH 7.5. The injection buffer was prepared on the pharmaceutical-grade water for intravenous administration from reagents of the molecular biology-grade purity (>99.5%), filter-sterilized, and stored at +4° C. until use.

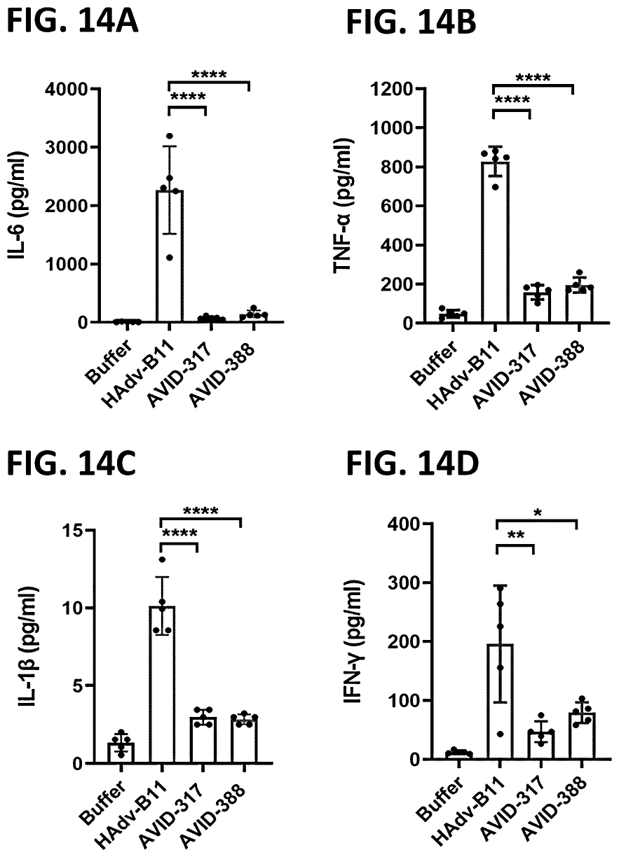

Mice were administered in groups of 2 (Control group), or >3 per experimental setting. Blood was collected through retro-orbital bleeding, and heparinized plasma was immediately prepared and stored at −80 C until subsequent analyzes were performed. The analysis of cytokine concentrations in plasma was perform using Bio-Rad Bio-Plex Pro™ Mouse Cytokine 23-Plex #M60009RDPD in accordance with manufacturer's instructions using 25 ml of plasma sample for each mouse. The results of tests of inflammatory cytokine concentrations in mouse plasma after administration of these viruses at a single dose of 1×10¹¹ per mouse in 200 ml of injection buffer are shown in FIG. 14A-14D.

This analysis demonstrated that the intravenous administration of the very high doses of AVID-317 and A VID-388 vectors of the invention, comprising mutated pentons and fibers, triggered release of significantly lower amounts of inflammatory cytokines in plasma, compared to HAdv-B11 virus with unmodified capsid.

### Example 4. CD46-Interacting Adenovirus Vectors are Cytotoxic to Human CD33-Positive Myeloid Cell Populations in Peripheral Blood

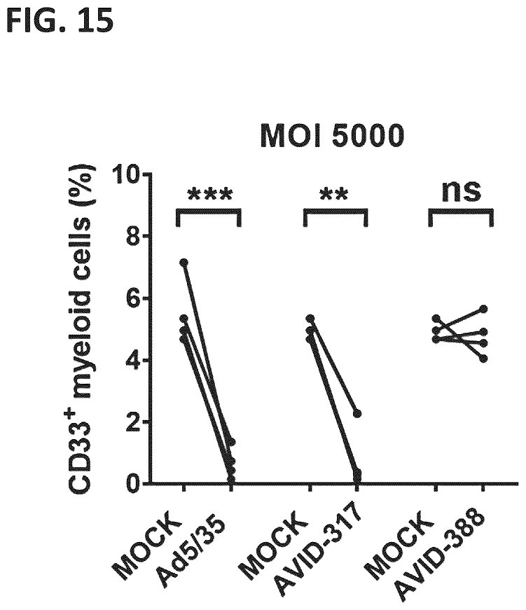

In clinical settings, intravenous injection will inevitably lead to exposure of circulating blood cells to the viral vectors. We sought to analyze whether and to what extent the engineered AVID-317 and AVID-388 vectors may reduce viability of mononuclear cell populations in human blood. To that end, we first infected primary human peripheral blood mononuclear cells (PBMCs) with a known in the art Ad5/35 vector, comprising mutated CD46-interacting fiber but comprising unmodified penton, and novel vectors of the invention, AVID-317 and AVID-388 vectors, at an MOI of 5000 v.p./cell. Infection of primary human blood mononuclear cells with these vectors led to a dramatic reduction and a near complete disappearance of CD33+ myeloid cells within 24 hours after cell infection with Ad5/35 and AVID-317 vectors comprising CD46-interacting fibers (FIG. 15). In contrast, the proportion of CD33-positive myeloid cells among human blood mononuclear cells have not declined after their infection with AVID-388 vector comprising DSG2-interacting fiber. (FIG. 15). Taken together, these data demonstrate that human CD33+ mononuclear cells in peripheral blood are highly sensitive to infection with CD46-interacting vectors. Exposure of CD33+ blood mononuclear cells to CD46-interacting vectors triggers cytotoxicity and disappearance of these cells from the PBMC population pool. In comparison, the DSG2-interacting AVID-388 vector was not able to trigger disappearance of CD33-positive human blood mononuclear cells, demonstrating that infection of these cells with a DSG2-interacting vector is not cytotoxic. This Example demonstrates that the adenovirus vectors of the invention, comprising DSG2-interacting fibers, are the preferred embodiments of the invention and a platform for therapeutic gene delivery to human cells in vivo, including LT-HSC cells, when low or no vector-induced cytotoxicity is desirable. In contrast, the adenovirus vectors of the invention, comprising CD46-interacting fibers may be preferred embodiments of the invention and a platform for applications, where the virus-induced cytotoxicity is desirable or clinically acceptable. Such applications include, without limitation, therapy of patients with cancer.

### Example 5. DSG2-Interacting a VID-388 Virus, Comprising Mutated Penton Base Protein, Efficiently Infects Primitive Human CD34+ Hematopoietic Cells In Vitro

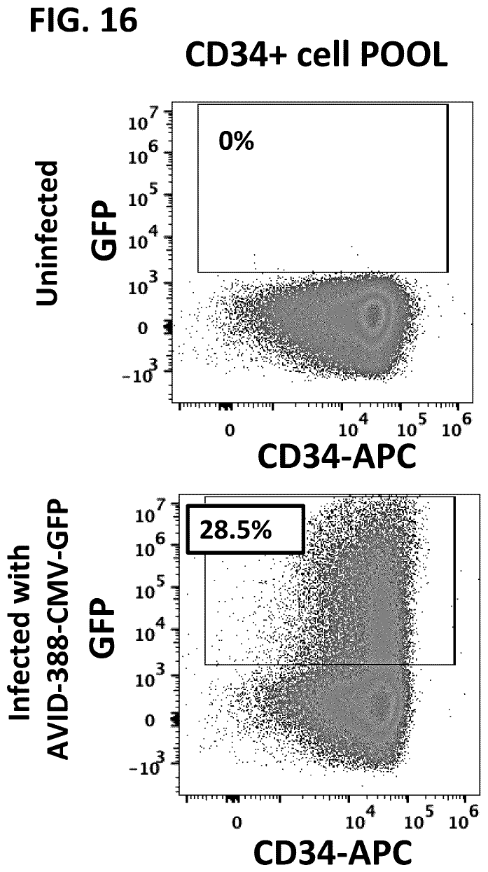

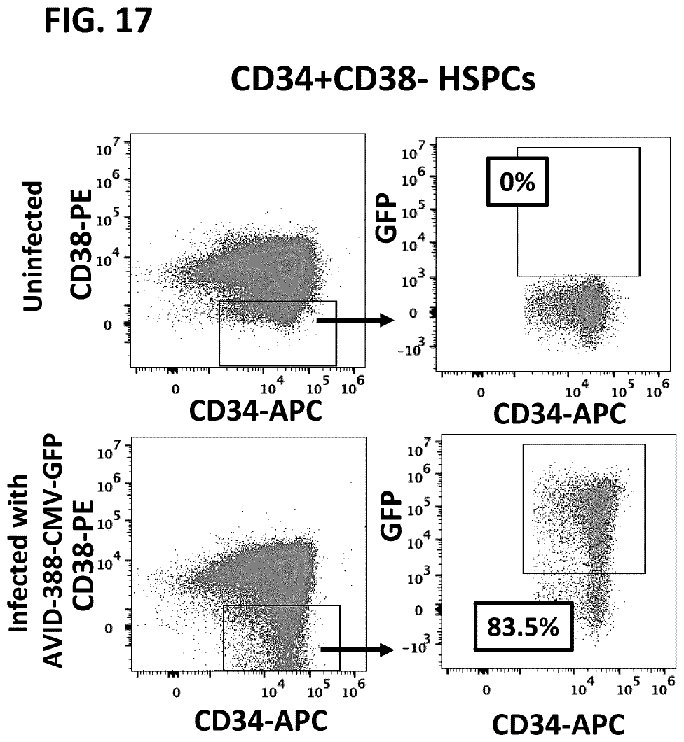

To determine whether capsid-mutated DSG2-interacting AVID-388-CMV-GFP vector is able to infect primary human CD34⁺ cells, we infected human CD34⁺ cells, isolated from mobilized peripheral blood-derived (supplied by Lonza), with AVID-388-CMV-GFP virus at an MOI of 5000 v.g. per cell in IMDM+15% FBS media, and percent of GFP-expressing cells was analyzed 48 h later by flow cytometry. Prior to analyzing the proportion of GFP-expressing cells, cells were stained with fluorescent APC-labeled anti-CD34 and PE-labeled anti-CD38 antibodies to allow for specific gating on total CD34⁺ cells as well as on the more primitive CD34-positive/CD38-negative cells (CD34⁺CD38⁻ cells). This analysis showed that 48 hours after the addition of the virus to cells, the proportion of CD34⁺ cells that expressed GFP reporter gene was 28.5% (FIG. 16), demonstrating that the AVID-388-CMV-GFP virus is rather efficient at gene delivery to primary human CD34⁺ cells. Specific gating on a more primitive CD34⁺CD38⁻ cells, however showed that 83.5% of this cell population expressed GFP after cell infection with DSG2-interacting AVID-388-CMV-GFP capsid-modified virus (FIG. 17).

### Example 6. DSG2-Interacting a VID-388-CMV-GFP Virus, Comprising Mutated Penton Base Protein, Efficiently Infects Primitive Human CD34+ Hematopoietic Cells In Vivo

For in vivo gene transfer analyses, we chose the commercially available humanized CD34⁺ hu-NSG™ mouse model, grafted with primary human CD34⁺ cells isolated from cord blood (Jackson Laboratories). Mice were conditioned with human G-CSF for 5 days to expand the HSPC compartment prior to AVID-388-CMV-GFP administration and were mobilized with AMD3100 three hours prior to virus administration. Mice were also conditioned with dexamethasone to further suppress the innate immune activation that inevitably occurs following intravenous administration of viral vectors. Forty-eight hours after intravenous administration of AVID-388-CMV-GFP or Buffer (control group), mice were sacrificed, and bone marrow was harvested for flow cytometry analyses. Due to the extreme paucity of HSPCs, harvested bone marrow was pooled from 3 to 7 mice per group prior to CD34⁺ cell enrichment, staining with antibodies to different cell markers, and flow cytometry analysis of distribution of GFP⁺ cells among various BM cell populations. The experiment was repeated 3 times.

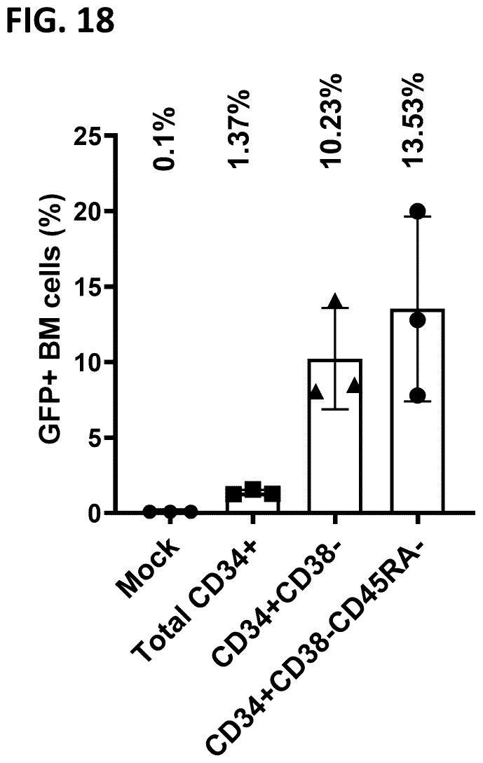

Analysis of the efficacy of in vivo gene delivery to human CD34⁺ cells in mouse bone marrow demonstrated that 1.37±0.18% of total human CD34⁺ cells in the bone marrow expressed GFP 48 h after intravenous administration of A VID-388-CMV-GFP (FIG. 18). The proportion of GFP⁺ cells increased to an average of 10.23±3.36% when gating on a more primitive CD34⁺CD38⁻ cell subset was applied. The efficacy of A VID-388-mediated in vivo gene delivery to primitive CD34⁺CD38⁻CD45RA⁻ bone marrow cells on averaged 13.53% and could reach up to 20% (FIG. 18).

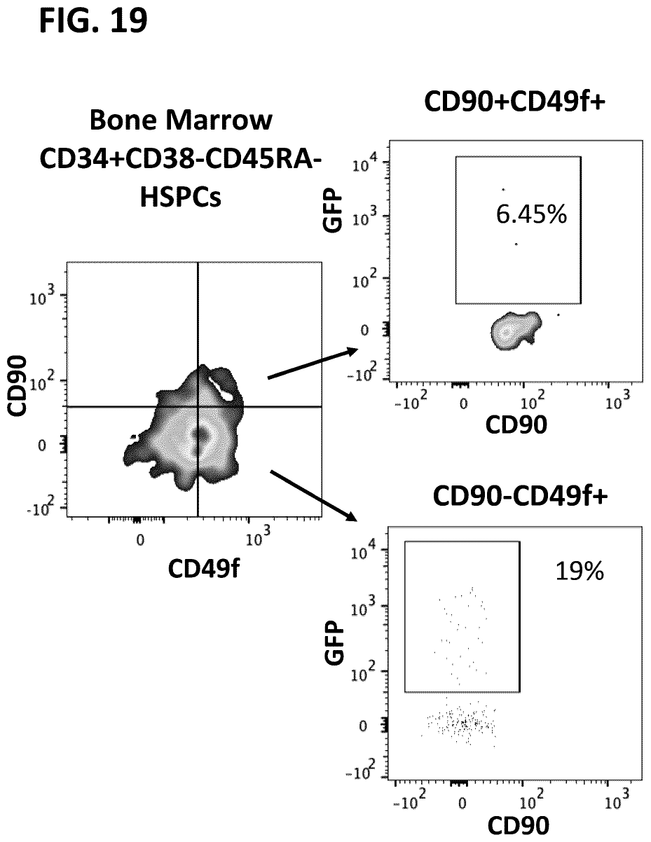

It is known in the art that long-term multilineage engraftment can be achieved by serial transplantation of primitive CD34⁺CD38⁻CD45RA⁻ cells that express CD49f (also known as □6-integrin) with or without CD90 expression. Because the penton within AVID-388 was engineered to utilize CD49f (FIG. 8 and SEQ ID NO:19) to mediate vector entry into cells, we added a CD49f-specific antibody to our antibody panel and analyzed gene transfer to CD90⁺CD49f⁺ and CD90⁻CD49f⁺ cell populations after intravenous AVID-388-CMV-GFP administration. This analysis demonstrated that after gating on primitive CD34⁺CD38⁻CD45RA⁻ cells, the expression of both CD90 and CD49f on bone marrow cells could be reliably detected (FIG. 19). Evaluation of the efficacy of gene delivery to the CD90⁺CD49f⁺ subset showed that 6.45% of these cells expressed GFP 48 h after intravenous administration of AVID-388-CMV-GFP, while the efficacy of in vivo gene transfer to the CD90⁻CD49f⁺ subset reached 19% (FIG. 19).

### Example 7. Capsid-Mutated a VID-388 Vector, Comprising DSG2-Interacting Fiber Knob Domain and Mutated Penton Base Protein, Exhibits Potent Anti-Tumor Activity after Intravenous Administration

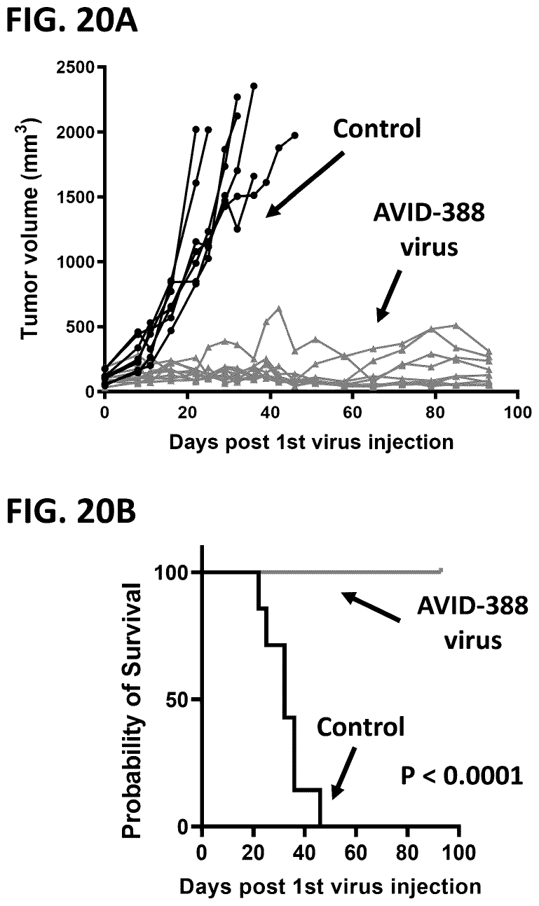

Next, we tested the anti-tumor potency of the DSG2-interacting AVID-388 vector platform in a pre-clinical model of localized cancer. Human lung adenocarcinoma A549 cells were grafted subcutaneously to NSG mice (NOD.Cg-Prkdcscid Il2rgtm1Wjl/SzJ; Jackson Laboratories strain #005557) at 5×10⁶ cells per site and, when tumor size reached about 125 mm³, mice were intravenously administered with three injections of replication-competent AVID-388, comprising adenovirus death protein in the E3 region, at a dose of 4×10¹⁰ v.p./mouse at 48-hour intervals or Buffer (Control group). The tumor growth kinetics were monitored for up to 90 days post virus administration. Mice were sacrificed when tumor volume reached 2000 mm³. This analysis demonstrated that intravenous administration of AVID-388 could efficiently suppress tumor growth (FIG. 20). While all tumor-bearing mice treated with Buffer succumbed to tumor growth and had to be sacrificed within 45 days after the beginning of treatment, all tumor-bearing mice that were treated with AVID-388 virus survived for the duration of the study (FIG. 20).

### Example 8. The Use of AVID-317 Therapeutic Platform for Treatment of Localized and Disseminated Cancer

Next, we utilized a replication-competent AVID-317 vector that comprises fiber that interacts with CD46 attachment receptor and further comprises mutated penton, to analyze the anti-tumor potency of this vector platform in pre-clinical models of localized and disseminated cancer using intravenous route of virus delivery. For these studies, the replication competent AVID-317 vector comprised adenovirus death protein in the E3 region of the virus.

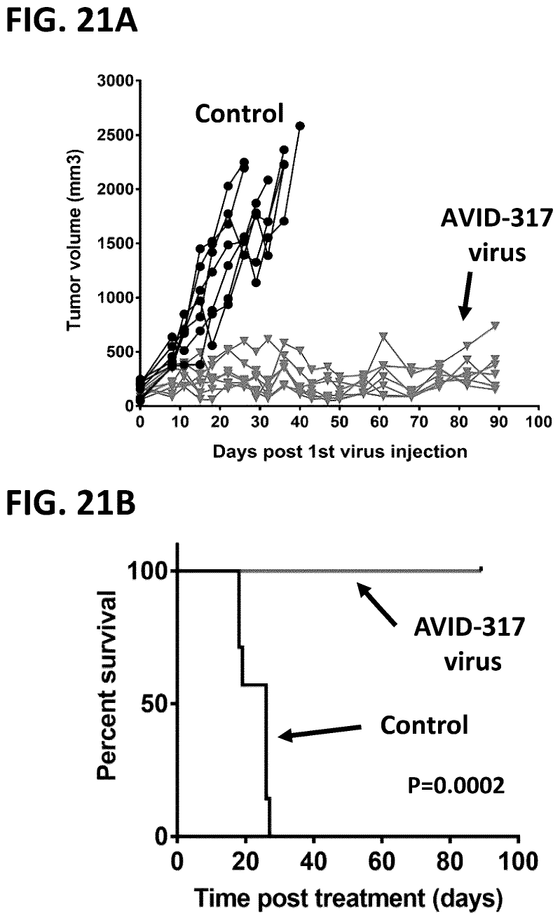

For pre-clinical localized cancer model, we utilized NSG mice (NOD.Cg-Prkdcscid Il2rgtm1Wjl/SzJ; Jackson Laboratories strain #005557) with subcutaneously grafted human lung adenocarcinoma A549 cells. A549 cells were grafted at 5×10⁶ cells per site and, when tumor size reached about 125 mm³, mice were intravenously administered with three injections of AVID-317 at a dose of 4×10¹⁰ v.p./mouse at 48-hour intervals or Buffer (Control group). The tumor growth kinetics were monitored for up to 90 days post virus administration. Mice were sacrificed when tumor volume reached 2000 mm³. This analysis demonstrated that while all tumor-bearing mice treated with buffer succumbed to tumor growth and had to be sacrificed within 40 days after the beginning of treatment, all tumor-bearing mice that were treated with AVID-317 virus survived for the duration of the study (FIG. 21). Importantly, the histological analyses of the tumor tissue at 90-day time point post virotherapy failed to reveal any tumor cell presence in about 50% of mice, indicating that the grafted tumor nodule was replaced by a scar tissue after treatment with AVID-317.

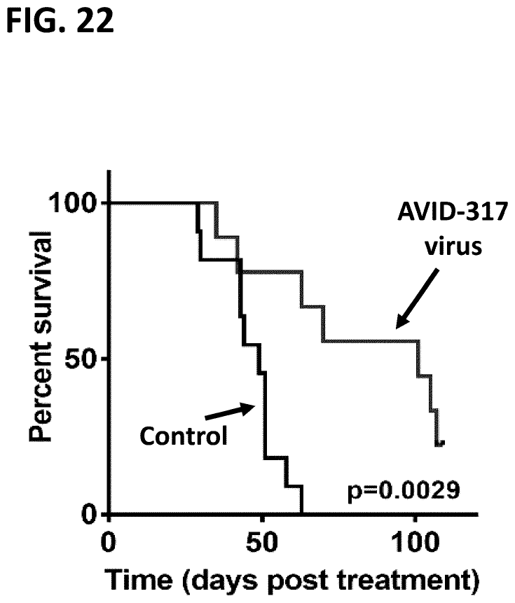

For disseminated lung cancer model, we utilized NCr-Nude mice (supplied by Taconic) that were grafted with human lung adenocarcinoma A549-Luc-C5 cells intravenously. Ten-to twelve-week-old female NCr-Nude mice were intravenously injected with 2.5×10⁶ A549-luc-C5 cells, which express firefly luciferase under the control of NF-kB-responsive promoter. Tumor burden in the lung was monitored using non-invasive IVIS imaging that measured relative luciferase unit (RLU) luminescence of tumor cells starting week 5 post tumor grafting. Eight to ten weeks after tumor transplantation, when the tumor burden reached a luminescence signal from 2 to 8×10⁶ RLU, mice were enrolled into treatment experiment and randomized between AVID-317-treated and Buffer-treated (Control) cohorts. In virus-treated cohort, mice were administered intravenously with three injections of AVID-317 at a dose of 5×10¹⁰ v.p./mouse at 48-hour intervals. In Buffer-treated cohorts, mice were administered with buffer with the same schedule of administration. The luminescence of tumors was monitored weekly for the up to 105 days, and mice were sacrificed when they experienced a 20% weight loss against their weight prior to virus treatment. The log-rank survival plot of the data obtained from this experiment is shown in FIG. 22. This analysis demonstrated that therapy of mice bearing disseminated tumors in the lungs with AVID-317 significantly extends their survival, compared to tumor-bearing mice treated with buffer (FIG. 22).

### Example 9. Generation of Novel Therapeutic Ad Vectors with Desired Multiplexed Receptor Specificities

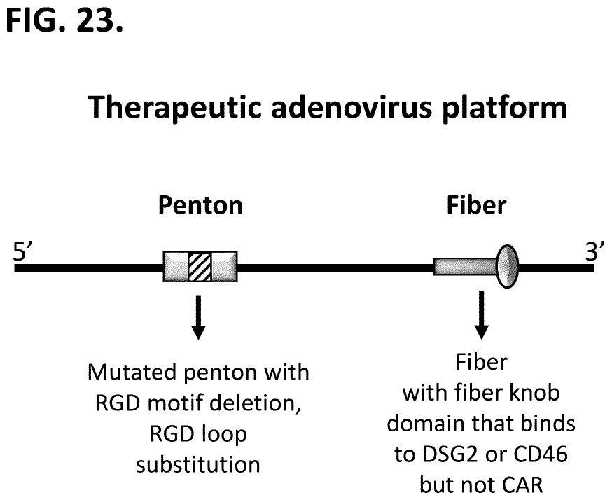

Using methodologies established in the art, one of the ordinary skills in the art can now construct novel Ad vectors with desired and restricted receptor specificities and targeted to specific human cell types by replacing artificial peptides interacting with a6-integrins within penton base proteins of AVID-317 or AVID-388 vectors, as shown in Examples 1 and 2, with peptides interacting with a4-, a9, or aD-integrin classes. Using methods established in the art, one of the ordinary skills in the art can replace the penton base open reading frame in the adenovirus vector genome for an open reading frame of a penton base protein, comprising peptide interacting with a4-integrins classes (SEQ ID NO:21) as shown in FIG. 9; or peptide interacting with a9-integrin classes (SEQ ID NO:23) as shown in FIG. 10; or peptide interacting with aD-integrin classes (SEQ ID NO:25) as shown in FIG. 11. When combined with fiber knob domain targeting to CD46 or DSG2 attachment receptors, but not CAR, such multiplexed targeted therapeutic adenovirus vectors (FIG. 23) may prove useful for targeted transduction of human cells ex vivo or in vivo after intravenous administration, when the RGD-integrin dependent cell transduction is undesirable due to virus-induced toxicity and broad off-target cell transduction.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary. The appendixes which are hereby incorporated by reference as if fully set forth herein.

### Sequences Cited in the Specification

SEQ ID NO: 1
Organism: Human laminin-1-derived peptide
SIKVAV
SEQ ID NO: 2
Organism: Human adenovirus type 5 fiber ACCESSION NO: AP_000226
MKRARPSEDT FNPVYPYDTE TGPPTVPFLT PPFVSPNGFQ ESPPGVLSLR LSEPLVTSNG
MLALKMGNGL SLDEAGNLTS QNVTTVSPPL KKTKSNINLE ISAPLTVTSE ALTVAAAAPL
MVAGNTLTMQ SQAPLTVHDS KLSIATQGPL TVSEGKLALQ TSGPLTTTDS STLTITASPP
LTTATGSLGI DLKEPIYTQN GKLGLKYGAP LHVTDDLNTL TVATGPGVTI NNTSLQTKVT
GALGFDSQGN MQLNVAGGLR IDSQNRRLIL DVSYPFDAQN QLNLRLGQGP LFINSAHNLD
INYNKGLYLF TASNNSKKLE VNLSTAKGLM FDATAIAINA GDGLEFGSPN APNTNPLKTK
IGHGLEFDSN KAMVPKLGTG LSFDSTGAIT VGNKNNDKLT LWTTPAPSPN CRLNAEKDAK
LTLVLTKCGS QILATVSVLA VKGSLAPISG TVQSAHLIIR FDENGVLLNN SFLDPEYWNF
RNGDLTEGTA YTNAVGFMPN LSAYPKSHGK TAKSNIVSQV YLNGDKTKPV TLTITLNGTQ
ETGDTTPSAY SMSFSWDWSG HNYINEIFAT SSYTFSYIAQ E
SEQ ID NO: 3
Organism: Human adenovirus type 11 fiber ACCESSION NO: AAA42490
MTKRVRLSDS FNPVYPYEDE STSQHPFINP GFISPNGFTQ SPNGVLTLKC LTPLTTTGGS
LQLKVGGGLT VDDTNGFLKE NISATTPLVK TGHSIGLPLG AGLGTNENKL CIKLGQGLTF
NSNNICIDDN INTLWTGVNP TEANCQIMNS SESNDCKLIL TLVKTGALVT AFVYVIGVSN
NFNMLTTHRN INFTAELFFD STGNLLTRLS SLKTPLNHKS GQNMATGAIT NAKGFMPSTT
AYPFNDNSRE KENYIYGTCY YTASDRTAFP IDISVMLNRR AINDETSYCI RITWSWNTGD
APEVQTSATT LVTSPFTFYY IREDD
SEQ ID NO: 4
Organism: Human adenovirus type 16 fiber ACCESSION NO: AET87329
MAKRARLSSS FNPVYPYEDE SSSQHPFINP GFISSNGFAQ SPDGVLTLKC VNPLTTASGP
LQLKVGSSLT VDTIDGSLEE NITAAAPLTK TNHSIGLSIG SGLQTKDDEL CLSLGDGLVT
KDDKLCLSLG DGLITKDDTL CAKLGHGLVF DSSNAITIEN NTLWTGAKPS ANCVIQEGED
SPDCKLTLVL VKNGGLINGY ITLMGASQYT NTLFKNKQVT INVNLAFDNT GQIITYLSSL
KSNLNFKDNQ NMATGTITSA KGFMPSTTAY PFTIYATQSL NEDYIYGECY YKSTNGTLFP
LKVTVTLNRR MSASGMAYAM NFSWSLNAEQ APETTEVTLI TSPFFFSYIR EDD
SEQ ID NO: 5
Organism: Human adenovirus type 21 fiber ACCESSION NO: AAW33370
MTKRVRLSDS FNPVYPYEDE STSQHPFINP GFISPNGFTQ SPDGVLTLNC LTPLTTTGGP
LQLKVGGGLI VDDTDGTLQE NIRATAPITK NNHSVELSIG NGLETQNNKL CAKLGNGLKF
NNGDICIKDS INTLWTGIKP PPNCQIVENT DTNDGKLTLV LVKNGGLVNG YVSLVGVSDT
VNQMFTQKSA TIQLRLYFDS SGNLLTDESN LKIPLKNKSS TATSEAATSS KAFMPSTTAY
PFNTTTRDSE NYIHGICYYM TSYDRSLVPL NISIMLNSRT ISSNVAYAIQ FEWNLNAKES
PESNIATLTT SPFFFSYIRE DDN
SEQ ID NO: 6
Organism: Human adenovirus type 34 fiber ACCESSION NO: AAW33501
MTKRVRLSDS FNPVYPYEDE STSQHPFINP GFISPNGFTQ SPDGVLTLKC LTPLTTTGGS
LQLKVGGGLT VDDTDGTLQE NIRATAPITK NNHSVELSIG NGLETQNNKL CAKLGNGLKF
NNGDICIKDS INTLWTGINP PPNCQIVENT NTNDGKLTLV LVKNGGLVNG YVSLVGVSDT
VNQMFTQKTA NIQLRLYFDS SGNLLTDESD LKIPLKNKSS TATSETVASS KAFMPSTTAY
PENTTTRDSE NYIHGICYYM TSYDRSLFPL NISIMLNSRM ISSNVAYAIQ FEWNLNASES
PESNIATLTT SPFFFSYITE DDN
SEQ ID NO: 7
Organism: Human adenovirus type 35 fiber ACCESSION NO: AP_000601
MTKRVRLSDS FNPVYPYEDE STSQHPFINP GFISPNGFTQ SPDGVLTLKC LTPLTTTGGS
LQLKVGGGLT VDDTDGTLQE NIRATAPITK NNHSVELSIG NGLETQNNKL CAKLGNGLKF
NNGDICIKDS INTLWTGINP PPNCQIVENT NTNDGKLTLV LVKNGGLVNG YVSLVGVSDT
VNQMFTQKTA NIQLRLYFDS SGNLLTEESD LKIPLKNKSS TATSETVASS KAFMPSTTAY
PFNTTTRDSE NYIHGICYYM TSYDRSLFPL NISIMLNSRM ISSNVAYAIQ FEWNLNASES
PESNIATLTT SPFFFSYITE DDN
SEQ ID NO: 8
Organism: Human adenovirus type 50 fiber ACCESSION NO: AAW33547
MTKRVRLSDS FNPVYPYEDE STSQHPFINP GFISPNGFTQ SPDGVLTLNC LTPLTTTGGP
LQLKVGGGLI VDDTDGTLQE NIRVTAPITK NNHSVELSIG NGLETQNNKL CAKLGNGLKF
NNGDICIKDS INTLWTGIKP PPNCQIVENT DTNDGKLTLV LVKNGGLVNG YVSLVGVSDT
VNQMFTQKSA TIQLRLYFDS SGNLLTDESN LKIPLKNKSS TATSEAATSS KAFMPSTTAY
PFNTTTRDSE NYIHGICYYM TSYDRSLVPL NISIMLNSRT ISSNVAYAIQ FEWNLNAKES
PESNIATLTT SPFFFSYIRE DDN
SEQ ID NO: 9
Organism: Human adenovirus type 3 fiber ACCESSION NO: ABB17809
MAKRARLSTS FNPVYPYEDE SSSQHPFINP GFISPDGFTQ SPNGVLSLKC VNPLTTASGS
LQLKVGSGLT VDTTDGSLEE NIKVNTPLTK SNHSINLPIG NGLQIEQNKL CSKLGNGLTF
DSSNSIALKN NTLWTGPKPE ANCIIEYGKQ NPDSKLTLIL VKNGGIVNGY VTLMGASDYV
NTLFKNKNVS INVELYFDAT GHILPDSSSL KTDLELKYKQ TADFSARGFM PSTTAYPFVL
PNAGTHNENY IFGQCYYKAS DGALFPLEVT VMLNKRLPDS RTSYVMTFLW SLNAGLAPET
TQATLITSPF TFSYIREDD
SEQ ID NO: 10
Organism: Human adenovirus type 7 fiber ACCESSION NO: QEQ50315
MTKRVRLSDS FNPVYPYEDE STSQHPFINP GFISPNGFTQ SPDGVLTLKC LTPLTTTGGS
LQLKVGGGLT IDDTDGFLKE NISATTPLVK TGHSIGLSLG PGLETNENKL CAKLGEGLTF
NSNNICINDN INTLWTGVNP TRANCQIMAS SESNDCKLIL TLVKTGALVT AFVYVIGVSN
DFNMLTTHKN INFTAELFFD STGNLLTSLS SLKTPLNHKS GQNMATGALT NAKGFMPSTT
AYPFNVNSRE KENYIYGTCY YTASDHTAFP IDTSVMLNQR ALNNETSYCI RVTWSWNTGV
APEVQTSATT LVTSPFTFYY IREDD
SEQ ID NO: 11
Organism: Human adenovirus type 14 fiber ACCESSION NO: ACO81814
MTKRVRLSDS FNPVYPYEDE STSQHPFINP GFISPNGFTQ SPDGVLTLKC LTPLTTTGGS
LQLKVGGGLT VDDTDGTLQE NIGATTPLVK TGHSIGLSLG AGLGTDENKL CTKLGEGLTF
NSNNICIDDN INTLWTGVNP TEANCQMMDS SESNDCKLIL TLVKTGALVT AFVYVIGVSN
NFNMLTTYRN INFTAELFFD SAGNLLTSLS SLKTPLNHKS GQNMATGAIT NAKSFMPSTT
AYPFNNNSRE NYIYGTCHYT ASDHTAFPID ISVMLNQRAI RADTSYCIRI TWSWNTGDAP
EGQTSATTLV TSPFTFYYIR EDD
SEQ ID NO: 12
Organism: Human adenovirus type 55 fiber ACCESSION NO: AQY04135
MTKRVRLSDS FNPVYPYEDE STSQHPFINP GFISPNGFTQ SPDGVLTLKC LTPLTTTGGS
LQLKVGGGLT VDDTDGTLQE NIGTTTPLVK TGHSIGLSLG AGLGTDENKL CTKLGKGLTF
NSNNICIDDN INTLWTGINP TEANCQMMDS SESNDCKLIL TLVKTGALVT AFVYVIGVSN
NFNMLTTYRN INFTAELFFD SAGNLLTSLS SLKTPLNHKS GQNMATGAIT NAKSFMPSTT
AYPFNNNSRE KENYIYGTCH YTASDHTAFP IDISVMLNQR AIRADTSYCI RITWSWNTGD
APEGQTSATT LVTSPFTFYY IREDD
SEQ ID NO: 13
Organism: Ad5/35 fiber, chimeric artificial
MKRARPSEDTFNPVYPYDTETGPPTVPFLTPPFVSPNGFQESPPGVLSLRLADPVTTKNG
EITLKLGEGVDLDDSGKLIANTVNKAIAPLSFSNNTISLNMDTPLYTKDGKLSLQVSPPLS
ILKSTILNTLALAFGSGLGLSGSALAVQLASPLTFDDKGNIKITLNRGLHVTTGDAIESNIS
WAKGIKFEDGAIATNIGKGLEFGTSSTETGVNNAYPIQVKLGSGLSFDSTGAIMAGNKD
YDKLTLWTGINPPPNCQIVENTNTNDGKLTLVLVKNGGLVNGYVSLVGVSDTVNQMFT
QKTANIQLRLYFDSSGNLLTDESDLKIPLKNKSSTATSETVASSKAFMPSTTAYPFNTTTR
DSENYIHGICYYMTSYDRSLFPLNISIMLNSRMISSNVAYAIQFEWNLNASESPESNIATLT
TSPFFFSYITEDDN
SEQ ID NO: 14
Organism: Ad5/14 fiber, chimeric artificial
Organism: Fiber mutant; Artificial
MKRARPSEDTFNPVYPYDTETGPPTVPFLTPPFVSPNGFQESPPGVLSLRLADPVTTKNG
EITLKLGEGVDLDDSGKLIANTVNKAIAPLSFSNNTISLNMDTPLYTKDGKLSLQVSPPLS
ILKSTILNTLALAFGSGLGLSGSALAVQLASPLTFDDKGNIKITLNRGLHVTTGDAIESNIS
WAKGIKFEDGAIATNIGKGLEFGTSSTETGVNNAYPIQVKLGSGLSFDSTGAIMAGNKD
YDKLTLWTGVNPTEANCQMMDSSESNDCKLILTLVKTGALVTAFVYVIGVSNNFNMLT
TYRNINFTAELFFDSAGNLLTSLSSLKTPLNHKSGQNMATGAITNAKSFMPSTTAYPFSN
NSREKENYIYGTCHYTASDHTAFPIDISVMLNQRAIRADTSYCIRITWSWNTGDAPEGQT
SATTLVTSPFTFYYIREDD
SEQ ID NO: 15
Organism: RGD peptide, artificial
RGD
SEQ ID NO: 16
Organism: Human adenovirus type 5 penton base RGD loop sequence, partial (FIG. 7A)
DHAIRGDTFAT
SEQ ID NO: 17
Organism: Human adenovirus type 5 penton ACCESSION NO: AAA42519
MRRAAMYEEG PPPSYESVVS AAPVAAALGS PFDAPLDPPF VPPRYLRPTG
GRNSIRYSEL APLFDTTRVY LVDNKSTDVA SLNYQNDHSN FLTTVIQNND
YSPGEASTQT INLDDRSHWG GDLKTILHTN MPNVNEFMFT NKFKARVMVS
RLPTKDNQVE LKYEWVEFTL PEGNYSETMT IDLMNNAIVE HYLKVGRQNG
VLESDIGVKF DTRNFRLGFD PVTGLVMPGV YTNEAFHPDI ILLPGCGVDF
THSRLSNLLG IRKRQPFQEG FRITYDDLEG GNIPALLDVD AYQASLKDDT
EQGGGGAGGS NSSGSGAEEN SNAAAAAMQP VEDMNDHAIRGDTFATRAEE
KRAEAEAAAE AAAPAAQPEV EKPQKKPVIK PLTEDSKKRS YNLISNDSTF
TQYRSWYLAY NYGDPQTGIR SWTLLCTPDV TCGSEQVYWS LPDMMQDPVT
FRSTRQISNF PVVGAELLPV HSKSFYNDQA VYSQLIRQFT SLTHVFNRFP
ENQILARPPA PTITTVSENV PALTDHGTLP LRNSIGGVQR VTITDARRRT
CPYVYKALGI VSPRVLSSRT F
SEQ ID NO: 18
Organism: Human laminin-1-derived peptide, artificial (FIG. 8A)
AASIKVAVSAA
SEQ ID NO: 19
Organism: Penton mutant; Artificial (FIG. 8B)
MRRAAMYEEGPPPSYESVVSAAPVAAALGSPFDAPLDPPFVPPRYLRPTGGRNSIRYSEL
APLFDTTRVYLVDNKSTDVASLNYQNDHSNFLTTVIQNNDYSPGEASTQTINLDDRSHW
GGDLKTILHTNMPNVNEFMFTNKFKARVMVSRLPTKDNQVELKYEWVEFTLPEGNYSE
TMTIDLMNNAIVEHYLKVGRQNGVLESDIGVKFDTRNFRLGFDPVTGLVMPGVYTNEA
FHPDIILLPGCGVDFTHSRLSNLLGIRKRQPFQEGFRITYDDLEGGNIPALLDVDAYQASL
KDDAASIKVAVSAAKKPVIKPLTEDSKKRSYNLISNDSTFTQYRSWYLAYNYGDPQTGI
RSWTLLCTPDVTCGSEQVYWSLPDMMQDPVTFRSTRQISNFPVVGAELLPVHSKSFYND
QAVYSQLIRQFTSLTHVFNRFPENQILARPPAPTITTVSENVPALTDHGTLPLRNSIGGVQ
RVTITDARRRTCPYVYKALGIVSPRVLSSRTF
SEQ ID NO: 20
Organism: Penton mutant; Artificial
MRRAAMYEEG PPPSYESVVS AAPVAAALGS PFDAPLDPPF VPPRYLRPTG
GRNSIRYSEL APLFDTTRVY LVDNKSTDVA SLNYQNDHSN FLTTVIQNND
YSPGEASTQT INLDDRSHWG GDLKTILHTN MPNVNEFMFT NKFKARVMVS
RLPTKDNQVE LKYEWVEFTL PEGNYSETMT IDLMNNAIVE HYLKVGRQNG
VLESDIGVKF DTRNFRLGFD PVTGLVMPGV YTNEAFHPDI ILLPGCGVDF
THSRLSNLLG IRKRQPFQEG FRITYDDLEG GNIPALLDVT AYQASLK
ETAASIKVAVSAAKKPVIK PLTEDSKKRS YNLISNDSTF TQYRSWYLAY
NYGDPQTGIR SWTLLCTPDV TCGSEQVYWS LPDMMQDPVT FRSTRQISNF
PVVGAELLPV HSKSFYNDQA VYSQLIRQFT SLTHVFNRFP ENQILARPPA
PTITTVSENV PALTDHGTLP LRNSIGGVQR VTITDARRRT CPYVYKALGI
VSPRVLSSRT F
SEQ ID NO: 21
Organism: Alpha4-integrin-interacting peptide, artificial (FIG. 9A)
AAEILDVPSTAA
SEQ ID NO: 22
Organism: Penton mutant; Artificial (FIG. 9B)
MRRAAMYEEGPPPSYESVVSAAPVAAALGSPFDAPLDPPFVPPRYLRPTGGRNSIRYSEL
APLFDTTRVYLVDNKSTDVASLNYQNDHSNFLTTVIQNNDYSPGEASTQTINLDDRSHW
GGDLKTILHTNMPNVNEFMFTNKFKARVMVSRLPTKDNQVELKYEWVEFTLPEGNYSE
TMTIDLMNNAIVEHYLKVGRQNGVLESDIGVKFDTRNFRLGFDPVTGLVMPGVYTNEA
FHPDIILLPGCGVDFTHSRLSNLLGIRKRQPFQEGFRITYDDLEGGNIPALLDVDAYQASL
KDDAAEILDVPSTAAKKPVIKPLTEDSKKRSYNLISNDSTFTQYRSWYLAYNYGDPQTGI
RSWTLLCTPDVTCGSEQVYWSLPDMMQDPVTFRSTRQISNFPVVGAELLPVHSKSFYND
QAVYSQLIRQFTSLTHVFNRFPENQILARPPAPTITTVSENVPALTDHGTLPLRNSIGGVQ
RVTITDARRRTCPYVYKALGIVSPRVLSSRTF
SEQ ID NO: 23
Organism: Alpha9-integrin-interacting peptide, artificial (FIG. 10A)
AAAEIDGIELAA
SEQ ID NO: 24
Organism: Penton mutant; Artificial (FIG. 10B)
MRRAAMYEEGPPPSYESVVSAAPVAAALGSPFDAPLDPPFVPPRYLRPTGGRNSIRYSEL
APLFDTTRVYLVDNKSTDVASLNYQNDHSNFLTTVIQNNDYSPGEASTQTINLDDRSHW
GGDLKTILHTNMPNVNEFMFTNKFKARVMVSRLPTKDNQVELKYEWVEFTLPEGNYSE
TMTIDLMNNAIVEHYLKVGRQNGVLESDIGVKFDTRNFRLGFDPVTGLVMPGVYTNEA
FHPDIILLPGCGVDFTHSRLSNLLGIRKRQPFQEGFRITYDDLEGGNIPALLDVDAYQASL
KDDAAAEIDGIELAAKKPVIKPLTEDSKKRSYNLISNDSTFTQYRSWYLAYNYGDPQTGI
RSWTLLCTPDVTCGSEQVYWSLPDMMQDPVTFRSTRQISNFPVVGAELLPVHSKSFYND
QAVYSQLIRQFTSLTHVFNRFPENQILARPPAPTITTVSENVPALTDHGTLPLRNSIGGVQ
RVTITDARRRTCPYVYKALGIVSPRVLSSRTF
SEQ ID NO: 25
Organism: AlphaD-integrin-interacting peptide, artificial (FIG. 11A)
AATQIDSPLNAA
SEQ ID NO: 26
Organism: Penton mutant; Artificial (FIG. 11B)
MRRAAMYEEGPPPSYESVVSAAPVAAALGSPFDAPLDPPFVPPRYLRPTGGRNSIRYSEL
APLFDTTRVYLVDNKSTDVASLNYQNDHSNFLTTVIQNNDYSPGEASTQTINLDDRSHW
GGDLKTILHTNMPNVNEFMFTNKFKARVMVSRLPTKDNQVELKYEWVEFTLPEGNYSE
TMTIDLMNNAIVEHYLKVGRQNGVLESDIGVKFDTRNFRLGFDPVTGLVMPGVYTNEA
FHPDIILLPGCGVDFTHSRLSNLLGIRKRQPFQEGFRITYDDLEGGNIPALLDVDAYQASL
KDDAATQIDSPLNAAKKPVIKPLTEDSKKRSYNLISNDSTFTQYRSWYLAYNYGDPQTGI
RSWTLLCTPDVTCGSEQVYWSLPDMMQDPVTFRSTRQISNFPVVGAELLPVHSKSFYND
QAVYSQLIRQFTSLTHVFNRFPENQILARPPAPTITTVSENVPALTDHGTLPLRNSIGGVQ
RVTITDARRRTCPYVYKALGIVSPRVLSSRTF
SEQ ID NO: 27
Organism: Shaft domain of the fiber protein, derived from human adenovirus serotype
HAdv-E4.
LSLRLADPVTTKNGEITLKLGEGVDLDDSGKLIANTVNKAIAPLSFSNNTISLNMDTPLYT
KDGKLSLQVSPPLSILKSTILNTLALAFGSGLGLSGSALAVQLASPLTFDDKGNIKITLNR
GLHVTTGDAIESNISWAKGIKFEDGAIATNIGKGLEFGTSSTETGVNNAYPIQVKLGSGLS
FDSTGAIMAGNKDYDKLTL
SEQ ID NO: 28
Organism: Alpha-4-integrin-interacting peptide, human fibronectin-derived
EILDVPST
SEQ ID NO: 29
Organism: Alpha-9-integrin-interacting peptide, artificial
AEIDGIEL
SEQ ID NO: 30
Organism: Alpha-D-integrin-interacting peptide, artificial
TQIDSPLN

What is claimed is:

1. A method for transducing human cells, comprising:
contacting human cells with a recombinant adenovirus, wherein the recombinant adenovirus comprises:
(a) a penton base protein, wherein the RGD amino acid motif is deleted; and
(b) a penton base protein further comprises an inserted peptide that lacks the RGD amino acid motif and said peptide interacts with non-RGD integrin classes, expressed on human cells; and
(c) a fiber protein capable of interacting with CD46 or DSG2 attachment receptors, but not with coxsackievirus and adenovirus receptor, CAR.

2. The method of claim 1, wherein the penton base protein comprises an inserted peptide capable of interacting with integrins of a4-classes, a6-classes, a9-classes, or aD-classes.

3. The method of claim 2, wherein the peptide is inserted into the RGD loop of the penton base protein.

4. The method of claim 3, wherein the peptide has the sequence selected from the group consisting of SEQ ID NO:1, SEQ ID NO:18, SEQ ID NO:21, SEQ ID NO:23, SEQ ID NO:25, SEQ ID NO:28, SEQ ID NO:29 and SEQ ID NO:30.

5. The method of claim 1, wherein the recombinant adenovirus is based on human or animal serotype and comprises a fiber knob domain capable of binding to a CD46 receptor.

6. The method of claim 5, wherein the fiber knob domain comprises SEQ ID NO:13.

7. The method of claim 1, wherein the recombinant adenovirus is based on human or animal serotype and comprises a fiber knob domain capable of binding to a DSG2 receptor.

8. A method of claim 7, wherein the fiber knob domain comprises SEQ ID NO:14.

9. The method of claim 1, wherein the recombinant adenovirus comprises one or several transgenes of interest and wherein the recombinant adenovirus is a replication-deficient, replication-restricted, or gutless adenovirus.

10. The method of claim 1, wherein the human cells are contacted with the recombinant adenovirus ex vitro.

11. The method of claim 1, wherein the human cells are contacted with the recombinant adenovirus in vivo.

12. A recombinant adenovirus, comprising:
(a) a penton base protein, wherein the RGD amino acid motif is deleted; and
(b) a penton base protein further comprises an inserted peptide that lacks the RGD amino acid motif and said peptide interacts with non-RGD integrin classes, expressed on human cells; and
(c) a fiber protein capable of interacting with CD46 or DSG2 attachment receptors, but not with coxsackievirus and adenovirus receptor (CAR).

13. A recombinant adenovirus of claim 12, wherein the penton base protein comprises an inserted peptide capable of interacting with integrins of a4-classes, a6-classes, a9-classes, or aD-classes.

14. A recombinant adenovirus of claim 13, wherein the peptide is inserted into the RGD loop of the penton base protein.

15. A recombinant adenovirus of claim 14, wherein the peptide has the sequence selected from the group consisting of SEQ ID NO:1, SEQ ID NO:18, SEQ ID NO:21, SEQ ID NO:23, SEQ ID NO:25, SEQ ID NO:28, SEQ ID NO:29, and SEQ ID NO:30.

16. The recombinant adenovirus of claim 12, wherein the recombinant adenovirus is based on human or animal serotype and comprises a fiber knob domain capable of binding to a CD46 receptor.

17. The recombinant adenovirus of claim 16, wherein the fiber knob domain comprises SEQ ID NO:13.

18. The recombinant adenovirus of claim 12, wherein the recombinant adenovirus is based on human or animal serotype and comprises a fiber knob domain capable of binding to a DSG2 receptor.

19. The recombinant adenovirus of claim 18, wherein the fiber knob domain comprises SEQ ID NO:14.

20. A pharmaceutical composition comprising:
a) the recombinant adenovirus of claim 12; and
b) a pharmaceutically acceptable carrier.
